SEQUENCE LISTING (29 sequences)

SEQ ID NO: 1 - protein, 8 aa, synthetic construct
  REGION 1..8: note = CDR sequence
GFTFNTYA

SEQ ID NO: 2 - protein, 10 aa, synthetic construct
  REGION 1..10: note = CDR sequence
IRSKYNNYAT

SEQ ID NO: 3 - protein, 16 aa, synthetic construct
  REGION 1..16: note = CDR sequence
VRHGNFGNSYVSWFAY

SEQ ID NO: 4 - protein, 9 aa, synthetic construct
  REGION 1..9: note = CDR sequence
TGAVTTSNY

SEQ ID NO: 5 - protein, 9 aa, synthetic construct
  REGION 1..9: note = CDR sequence
ALWYSNLWV

SEQ ID NO: 6 - protein, 125 aa, synthetic construct
  REGION 1..125: note = Heavy chain variable region sequence l
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 7 - protein, 109 aa, synthetic construct
  REGION 1..109: note = Light chain variable region sequence
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 8 - protein, 8 aa, synthetic construct
  REGION 1..8: note = CDR sequence
GFTFHDYA

SEQ ID NO: 9 - protein, 8 aa, synthetic construct
  REGION 1..8: note = CDR sequence
ISWNSGTI

SEQ ID NO: 10 - protein, 15 aa, synthetic construct
  REGION 1..15: note = CDR sequence
AKDIQYGNYYYGMDV

SEQ ID NO: 11 - protein, 6 aa, synthetic construct
  REGION 1..6: note = CDR sequence
QSVSSY

SEQ ID NO: 12 - protein, 9 aa, synthetic construct
  REGION 1..9: note = CDR sequence
QQRSNWPIT

SEQ ID NO: 13 - protein, 122 aa, synthetic construct
  REGION 1..122: note = Heavy chain variable region sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 14 - protein, 107 aa, synthetic construct
  REGION 1..107: note = Light chain variable region sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 15 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 18 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 19 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 20 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 21 - protein, 106 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 107 aa, Homo sapiens [sequence omitted]

SEQ ID NO: 24 - protein, 454 aa, synthetic construct
  REGION 1..454: note = Antibody heavy chain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 25 - protein, 215 aa, synthetic construct
  REGION 1..215: note = Antibody light chain
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 451 aa, synthetic construct
  REGION 1..451: note = Antibody heavy chain
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 27 - protein, 214 aa, synthetic construct
  REGION 1..214: note = Antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 29 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

(19) United States
(12) Patent Application Publication
(10) Pub. No.: US 2024/0368297 A1
(43) Pub. Date: Nov. 7, 2024

(54) METHODS OF TREATING LYMPHOMA WITH BISPECIFIC ANTIBODIES AGAINST CD3 AND CD20

(71) Applicant: Genmab A/S, Valby (DK)

(72) Inventors: Tommy R. LI, Plainsboro, NJ (US); Craig J. THALHAUSER, Plainsboro, NJ (US)

(21) Appl. No.: 18/633,939

(22) Filed: Apr. 12, 2024

Related U.S. Application Data
(60) Provisional application No. 63/459,104, filed on Apr. 13, 2023.

(51) Int. Cl. C07K 16/28; A61K 31/138; A61K 31/573; A61K 39/00; A61P 35/00

(52) U.S. Cl. CPC C07K 16/2887; A61K 31/138; A61K 31/573; A61P 35/00; C07K 16/2809; A61K 2039/545; C07K 2317/31

(57) ABSTRACT

The present invention provides dosing regimens of bispecific antibodies targeting both CD3 and CD20 when used in the treatment of lymphoma, such as B-cell Non-Hodgkin lymphoma (B-NHL).

### FIELD

The present invention relates to bispecific antibodies targeting both CD3 and CD20 and the use of such antibodies for use in the treatment of lymphoma, such as B-cell Non-Hodgkin lymphoma (B-NHL), for example, aggressive or indolent lymphoma. Advantageous treatment regimens are also provided.

### SEQUENCE LISTING

The instant application contains a Sequence Listing which has been submitted electronically in xml format and is hereby incorporated by reference in its entirety. Said xml copy, created on Apr. 11, 2024 is named GMI_227_Sequence_Listing.xml and is 39,329 bytes in size.

### BACKGROUND

Monoclonal antibodies (mAbs) have been shown to be highly successful for the treatment of cancer. A further promising approach to improve antibody therapy is by recruiting T cells specifically to the antigen-expressing cancer cells. This can be achieved by utilizing bsAbs targeting both T cells and antigen-expressing cells. However, initial clinical studies were rather disappointing mainly due to low efficacy, severe adverse effects (cytokine storm) and immunogenicity of the bispecific antibodies. Advances in the design and application of bispecific antibodies have partially overcome the initial barrier of cytokine release syndrome and improved clinical effectiveness without dose-limiting toxicities (Garber, 2014, Nat. Rev. Drug Discov. 13:799-801).

The CD20 molecule, also called human B-lymphocyte-restricted differentiation antigen or Bp35, is found on the surface of greater than 90% of B cells from peripheral blood or lymphoid organs and is expressed during early pre-B cell development and remains until plasma cell differentiation. CD20 is present on both normal B cells as well as malignant B cells. In particular, CD20 is expressed on greater than 90% of B cell non-Hodgkin lymphomas, but is not found on hematopoietic stem cells, pro-B cells, normal plasma cells, or other normal tissues. Methods for treating cancer as well as autoimmune and immune diseases by targeting CD20 are known in the art.

For example, the chimeric CD20 antibody rituximab has been used for or suggested for use in treating cancers such as aggressive or indolent lymphoma and chronic lymphocytic leukemia (CLL). The human monoclonal anti-CD20 antibody ofatumumab has been used for or suggested for use in treating among others various CLL indications, follicular lymphoma (FL), neuromyelitis optica (NMO), diffuse and relapsing-remitting multiple sclerosis (RRMS).

Currently, bispecific antibodies are under development that target both CD20 and CD3. For example, WO2011028952 describes amongst others the generation of CD3×CD20 bispecific molecules using Xencor's XmAb bispecific Fc domain technology, WO2014047231 describes REGN1979 and other CD3×CD20 bispecific antibodies generated using the FcΔAdp technology from Regeneron Pharmaceuticals, and Sun et al. (2015, Science Translational Medicine 7, 287ra70) describe a B cell-targeting anti-CD20/CD3 T cell-dependent bispecific antibody constructed using “knobs-into-holes” technology. Such bispecific antibodies are currently being tested in clinical trials for specific indications in humans.

A bispecific antibody of particular interest that is under development is epcoritamab (Duobody CD3×CD20; GEN3013) (Engelberts et al., 2020, EBioMedicine, Vol. 52, 102625, WO2016110576, and WO2019155008, incorporated herein by reference). Epcoritamab has been shown to trigger potent T-cell-mediated killing of CD20-expressing cells

It also has been shown that compounds and drugs engaging with T cells as the mode of action draw attention to cytokine release syndrome (CRS) as a frequent adverse event (AE); CRS includes fever with or without hypotension, hypoxia, and other symptoms.

Accordingly, novel and effective therapies which reduce CRS and other adverse events associated with administration of CD3×CD20 bispecific antibodies are needed.

### SUMMARY

Provided herein are methods of treating human subjects who have lymphoma such as B-cell Non-Hodgkin lymphoma (B-NHL), for example, an aggressive or indolent form of lymphoma, by administering a bispecific antibody which binds to CD3 and CD20, such as epcoritamab, by utilizing advantageous clinical treatment regimens.

In one aspect, provided herein is a method of treating B-NHL, including aggressive or indolent lymphoma, in a human subject, the method comprising administering to the subject a bispecific antibody, wherein the bispecific antibody comprises: (i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and (ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14, wherein the bispecific antibody is administered in a 28 day cycle comprising administering a first priming dose on day 1 of about 0.05 mg to about 0.35 mg, a first intermediate dose on about day 8 of about 0.6 mg to 5 mg, a second intermediate dose on about day 15 of about 1 mg to about 10 mg, followed by one weekly dose of between about 20-100 mg.

Alternatively, the bispecific antibody is administered in a 35 day cycle comprising administering a first priming dose on day 1 of about 0.05 mg to about 0.35 mg, a first intermediate dose on about day 8 of about 0.6 mg to 5 mg, a second intermediate dose on about day 15 of about 1 mg to about 10 mg, followed by at least two weekly doses of between about 20-100 mg.

In some embodiments, the priming dose is about 0.16 mg. In some embodiments, the first intermediate dose is about 0.8 mg. In some embodiments, the second intermediate dose is about 3 mg. In some embodiments, the second intermediate dose is about 6 mg.

In one embodiment, the method further comprises administering the bispecific antibody once every week at a dose of between about 24 to about 48 mg, for at least three weeks. In some embodiments, the method comprises administering the bispecific antibody once every week at a dose of between about 24 to about 48 mg, for at least four weeks. In some embodiments, the method comprises administering the bispecific antibody once every week at a dose of between about 24 to about 48 mg, for at least five weeks, at least six weeks, at least seven weeks or at least 8 weeks.

In some embodiments, the method further comprises administering the bispecific once every two weeks after the weekly administration (biweekly administration), e.g., for six 28-day cycles. In some embodiments, the method further comprises administering the bispecific antibody once every four weeks after the biweekly administration, e.g., for at least two 28-day

In some embodiments, the bispecific antibody is administered as follows:
(i) in cycle 1, a first priming dose of 0.16 mg is administered on day 1, a first intermediate dose of 0.8 mg is administered on day 8, a second intermediate dose of 3 mg is administered on day 15, and a dose of 24 mg is administered on day 22 and optionally on day 28
(ii) in cycles 2 and 3, a dose of 24 mg is administered on days 1, 8, 15, and 22
(iii) in cycles 4-9, a dose of 24 mg is administered on days 1 and 15; and
(iv) in cycle 10 and subsequent cycles a dose of 24 mg is administered on day 1.

In some embodiments, the bispecific antibody is administered as follows:
(i) in cycle 1, a first priming dose of 0.16 mg is administered on day 1, a first intermediate dose of 0.8 mg is administered on day 8, a second intermediate dose of 3 mg is administered on day 15, and a dose of 48 mg is administered on day 22 and optionally on day 28
(ii) in cycles 2 and 3, a dose of 48 mg is administered on days 1, 8, 15, and 22
(iii) in cycles 4-9, a dose of 48 mg is administered on days 1 and 15; and
(iv) in cycle 10 and subsequent cycles a dose of 48 mg is administered on day 1.

In some embodiments, the bispecific antibody is administered as follows:
(i) in cycle 1, a first priming dose of 0.16 mg is administered on day 1, a first intermediate dose of 0.8 mg is administered on day 8, a second intermediate dose of 6 mg is administered on day 15, and a dose of 24 mg is administered on day 22 and optionally on day 28
(ii) in cycles 2 and 3, a dose of 24 mg is administered on days 1, 8, 15, and 22
(iii) in cycles 4-9, a dose of 24 mg is administered on days 1 and 15; and
(iv) in cycle 10 and subsequent cycles a dose of 24 mg is administered on day 1.

In some embodiments, the bispecific antibody is administered as follows:
(i) in cycle 1, a first priming dose of 0.16 mg is administered on day 1, a first intermediate dose of 0.8 mg is administered on day 8, a second intermediate dose of 6 mg is administered on day 15, and a dose of 48 mg is administered on day 22 and optionally on day 28
(ii) in cycles 2 and 3, a dose of 48 mg is administered on days 1, 8, 15, and 22
(iii) in cycles 4-9, a dose of 48 mg is administered on days 1 and 15; and
(iv) in cycle 10 and subsequent cycles a dose of 48 mg is administered on day 1.

In some embodiments, the lymphoma is follicular lymphoma (FL), cutaneous T-cell lymphoma (CTCL), marginal zone lymphoma (MZL), chronic lymphocytic leukemia (CLL) or small cell lymphocytic lymphoma (SLL).

In one embodiment, the lymphoma is follicular lymphoma (FL).

In some embodiments, the subject is treated with prophylaxis for cytokine release syndrome (CRS). In some embodiments, the prophylaxis comprises administering a corticosteroid (e.g., dexamethasone at a dose of, e.g., 15 mg or equivalent thereof, including oral dose) on, for example, the same day as the bispecific antibody. In some embodiments, the corticosteroid is further administered on the second, third, and fourth days after administering the bispecific antibody.

In some embodiments, the subject is administered premedication, such as antihistamine (e.g., diphenhydramine, intravenously or orally at a dose of, e.g., 50 mg or equivalent thereof) and/or antipyretic (e.g., acetaminophen at a dose of, e.g., 560-1000 mg), to reduce reactions to injections. In some embodiments, the premedication is administered on the same day as the bispecific antibody.

In some embodiments, the prophylaxis and premedication are administered during cycle 1. In some embodiments, the prophylaxis is administered during cycle 2 when the subject experiences CRS greater than grade 1 after the last administration of the bispecific antibody in cycle 1. In some embodiments, the prophylaxis is continued in a subsequent cycle, when in the last administration of the bispecific antibody of the previous cycle, the subject experiences CRS greater than grade 1. In a further embodiment, the premedication is administered during cycle 2. In yet a further embodiment, the premedication is administered during subsequent cycles.

In some embodiments, the subject is administered antibiotics if the subject develops Grade 1 CRS. In some embodiments, the subject is administered a vasopressor if the subject develops Grade 2 or Grade 3 CRS. In some embodiments, the subject is administered at least two vasopressors if the subject develops Grade 4 CRS.

In some embodiments, the subject is administered tocilizumab if the subject develops Grade 2, Grade 3, or Grade 4 CRS. In some embodiments, the subject is further administered a steroid (e.g., dexamethasone or methylprednisolone). In some embodiments, tocilizumab is switched to an anti-IL-6 antibody (e.g., siltuximab) or an IL-IR antagonist (e.g., anakinra) if the subject is refractory to tocilizumab.

In some embodiments, the subject is administered prophylaxis for tumor lysis syndrome (TLS). In some embodiments, the prophylaxis for TLS comprises administering one or more uric acid reducing agents prior to administration of the bispecific antibody. In some embodiments, rasburicase and/or allopurinol is administered as the uric acid reducing agent. In some embodiments, when a subject shows signs of TLS, supportive therapy, such as rasburicase, may be used.

In some embodiments, the subject treated with the methods described herein achieves a complete response, a partial response, or stable disease, e.g., as defined by the Lugano criteria or LYRIC.

In some embodiments, the first antigen-binding region of the bispecific antibody comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and the second antigen-binding region comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.

In some embodiments, the first antigen-binding region of the bispecific antibody comprises a VH region comprising the amino acid sequence of SEQ ID NO: 6, and the VL region comprising the amino acid sequence of SEQ ID NO: 7; and the second antigen-binding region comprises a VH region comprising the amino acid sequence of SEQ ID NO: 13, and the VL region comprising the amino acid sequence of SEQ ID NO: 14.

In some embodiments, the first binding arm of the bispecific antibody is derived from a humanized antibody, preferably from a full-length IgG1,λ (lambda) antibody (e.g., SEQ ID NO: 22). In some embodiments, the second binding arm of the bispecific antibody is derived from a human antibody, preferably from a full-length IgG1,κ (kappa) antibody (e.g., SEQ ID NO: 23). In some embodiments, the bispecific antibody is a full-length antibody with a human IgG1 constant region.

In some embodiments, the bispecific antibody comprises an inert Fc region, for example, an Fc region in which the amino acids in the positions corresponding to positions L234, L235, and D265 in the human IgG1 heavy chain constant region of SEQ ID NO: 15 are F, E, and A, respectively. In some embodiments, the bispecific antibody comprises substitutions which promote bispecific antibody formation, for example, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa. The amino acid positions in SEQ ID NO: 15 correspond to the amino acid positions in in a human IgG1 heavy chain according to the Eu numbering scheme (Edelman et al., PNAS. 1969; 63:78-85; Kabat et al., Sequences of Proteins of Immunological Interest, Fifth Edition. 1991 NIH Publication No. 91-3242).

In some embodiments, the bispecific antibody has both an inert Fc region (e.g., substitutions at L234, L235, and D265 (e.g., L234F, L235E, and D265A)) and substitutions which promote bispecific antibody formation (e.g., F405L and K409R). In a further embodiment, the bispecific antibody comprises heavy chain constant regions comprising the amino acid sequences of SEQ ID NOs: 19 and 20.

In some embodiments, the bispecific antibody comprises a first heavy chain and a first light chain comprising (or consisting of) the amino acid sequences set forth in SEQ ID NOs: 24 and 25, respectively, and a second heavy chain and a second light chain comprising (or consisting of) the amino acid sequences set forth in SEQ ID NOs: 26 and 27, respectively. In some embodiments, the bispecific antibody is epcoritamab, or a biosimilar thereof.

### BRIEF DESCRIPTION OF THE DRAWINGS

FIG. 1 is a schematic of the Repeated Time-to-Event Model (RTTE). The hazard of repeated CRS AEs was described using a combination of nonlinear transformation of epcoritamab plasma concentration (ie, cytokine production) and an indirect effect compartment, varying between O and 1, to describe tolerance onset. Production in the indirect effect compartment was inhibited by epcoritamab plasma concentration.

FIG. 2 is a schematic of the Model-Predicted Grade 2+ CRS risk under a range of SUD regimens. Probability of grade 2+ CRS events during the first 2 cycles was simulated using a grid of 20 priming doses and 20 intermediate doses. The current SUD regimen (0.16/0.8/48 mg) is shown as a circle.

FIG. 3 is a graph depicting the Model-Predicted 2+ CRS risk under 3-step SUD regimens. Probability of at least one grade 2+ event. Full dose was repeated to complete 2 full cycles. The model-predicted probability of a patient experiencing at least 1 grade 2+ CRS event during the first 2 cycles is represented by solid circles (mean) and error bars (90% model prediction interval). The current SUD regimen (0.16/0.8/48 mg) is highlighted.

### DETAILED DESCRIPTION

The term “immunoglobulin” as used herein refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized (see, e.g., Fundamental Immunology Ch. 7 (Paul, W., ed., 2nd ed. Raven Press, N.Y. (1989)). Briefly, each heavy chain typically is comprised of a heavy chain variable region (abbreviated herein as VH or V_{H}) and a heavy chain constant region (abbreviated herein as CH or C_{H}). The heavy chain constant region typically is comprised of three domains, CH1, CH2, and CH3. The hinge region is the region between the CH1 and CH2 domains of the heavy chain and is highly flexible. Disulfide bonds in the hinge region are part of the interactions between two heavy chains in an IgG molecule. Each light chain typically is comprised of a light chain variable region (abbreviated herein as VL or V_{L}) and a light chain constant region (abbreviated herein as CL or C_{L}). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J Mol Biol 1987; 196:90117). Unless otherwise stated or contradicted by context, CDR sequences herein are identified according to IMGT rules (Brochet X., Nucl Acids Res 2008; 36: W503-508; Lefranc M P., Nucl Acids Res 1999; 27:209-12; www.imgt.org/). Unless otherwise stated or contradicted by context, reference to amino acid positions in the constant regions is according to the Eu-numbering (Edelman et al., PNAS. 1969; 63:78-85; Kabat et al., Sequences of Proteins of Immunological Interest, Fifth Edition. 1991 NIH Publication No. 91-3242). For example, SEQ ID NO: 15 sets forth amino acids positions 118-447, according to EU numbering, of the IgG1 heavy chain constant region.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is within the ability of one of ordinary skill in the art to align a sequence or segment in a sequence and thereby determine the corresponding position in a sequence to an amino acid position according to the present invention.

The term “antibody” (Ab) as used herein in the context of the present invention refers to an immunoglobulin molecule which has the ability to specifically bind to an antigen under typical physiological conditions with a half-life for a period of time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term antibody, unless specified otherwise, also encompasses polyclonal antibodies, monoclonal antibodies (mAbs), antibody-like polypeptides, chimeric antibodies and humanized antibodies.

The term “antibody fragment” or “antigen-binding fragment” as used herein refers to a fragment of an immunoglobulin molecule which retains the ability to specifically bind to an antigen, and can be generated by any known technique, such as enzymatic cleavage, peptide synthesis, and recombinant techniques. Examples of antibody fragments include (i) a Fab′ or Fab fragment, a monovalent fragment consisting of the VL, VH, CL and CH1 domains, or a monovalent antibody as described in WO2007059782 (Genmab); (ii) F (ab′) 2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 1989; 341:54446), which consists essentially of a VH domain and also called domain antibodies (Holt et al; Trends Biotechnol 2003; 21:484-90); (vi) camelid or nanobodies (Revets et al; Expert Opin Biol Ther 2005; 5:111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see, e.g., Bird et al., Science 1988; 242:42326 and Huston et al., PNAS 1988; 85:587983). Such single chain antibodies are encompassed within the term antibody fragment unless otherwise noted or clearly indicated by context.

The term “antibody-binding region” or “antigen-binding region” as used herein refers to the region which interacts with the antigen and comprises both the VH and the VL regions. The term antibody when used herein refers not only to monospecific antibodies, but also multispecific antibodies which comprise multiple, such as two or more, e.g., three or more, different antigen-binding regions. The term antigen-binding region, unless otherwise stated or clearly contradicted by context, includes fragments of an antibody that are antigen-binding fragments, i.e., retain the ability to specifically bind to the antigen.

As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g., IgG1, is mentioned, the term is not limited to a specific isotype sequence, e.g., a particular IgG1 sequence, but is used to indicate that the antibody is closer in sequence to that isotype, e.g., IgG1, than to other isotypes. Thus, e.g., an IgG1 antibody may be a sequence variant of a naturally occurring IgG1 antibody, which may include variations in the constant regions.

The term “bispecific antibody” or “bs” or “bsAb” as used herein refers to an antibody having two different antigen-binding regions defined by different antibody sequences. A bispecific antibody can be of any format.

The terms “half molecule”, “Fab-arm”, and “arm”, as used herein, refer to one heavy chain-light chain pair.

When a bispecific antibody is described as comprising a half-molecule antibody “derived from” a first parental antibody, and a half-molecule antibody “derived from” a second parental antibody, the term “derived from” indicates that the bispecific antibody was generated by recombining, by any known method, said half-molecules from each of said first and second parental antibodies into the resulting bispecific antibody. In this context, “recombining” is not intended to be limited by any particular method of recombining and thus includes all of the methods for producing bispecific antibodies described herein, including for example recombining by half-molecule exchange (also known as “controlled Fab-arm exchange”), as well as recombining at nucleic acid level and/or through co-expression of two half-molecules in the same cells.

The term “full-length” as used herein in the context of an antibody indicates that the antibody is not a fragment but contains all of the domains of the particular isotype normally found for that isotype in nature, e.g., the VH, CH1, CH2, CH3, hinge, VL and CL domains for an IgG1 antibody. A full-length antibody may be engineered. An example of a “full-length” antibody is epcoritamab.

The term “Fc region” as used herein refers to an antibody region consisting of the Fc sequences of the two heavy chains of an immunoglobulin, wherein said Fc sequences comprise at least a hinge region, a CH2 domain, and a CH3 domain.

The term “heterodimeric interaction between the first and second CH3 regions” as used herein refers to the interaction between the first CH3 region and the second CH3 region in a first-CH3/second-CH3 heterodimeric protein.

The term “homodimeric interactions of the first and second CH3 regions” as used herein refers to the interaction between a first CH3 region and another first CH3 region in a first-CH3/first-CH3 homodimeric protein and the interaction between a second CH3 region and another second CH3 region in a second-CH3/second-CH3 homodimeric protein.

As used herein, the term “binding” in the context of the binding of an antibody to a predetermined antigen typically is a binding with an affinity corresponding to a K_{D} of about 10-6 M or less, e.g. 10-7 M or less, such as about 10-8 M or less, such as about 10-9 M or less, about 10-10 M or less, or about 10-11 M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “isolated antibody” as used herein refers to an antibody which is substantially free of other antibodies having different antigenic specificities. In a preferred embodiment, an isolated bispecific antibody that specifically binds to CD20 and CD3 is in addition substantially free of monospecific antibodies that specifically bind to CD20 or CD3.

The term “CD3” as used herein refers to the human Cluster of Differentiation 3 protein which is part of the T-cell co-receptor protein complex and is composed of four distinct chains. CD3 is also found in other species, and thus, the term “CD3” is not limited to human CD3 unless contradicted by context. In mammals, the complex contains a CD3γ (gamma) chain (human CD3γ chain UniProtKB/Swiss-Prot No P09693, or cynomolgus monkey CD3Y UniProtKB/Swiss-Prot No Q95LI7), a CD3δ (delta) chain (human CD3δ UniProtKB/Swiss-Prot No P04234, or cynomolgus monkey CD3δ UniProtKB/Swiss-Prot No Q95LI8), two CD3ε (epsilon) chains (human CD3ε UniProtKB/Swiss-Prot No P07766, SEQ ID NO: 28); cynomolgus CD3ε UniProtKB/Swiss-Prot No Q95LI5; or rhesus CD3ε UniProtKB/Swiss-Prot No G7NCB9), and a CD3ζ-chain (zeta) chain (human CD3ζ UniProtKB/Swiss-Prot No P20963, cynomolgus monkey CD3ζ UniProtKB/Swiss-Prot No Q09TK0). These chains associate with a molecule known as the T-cell receptor (TCR) and generate an activation signal in T lymphocytes. The TCR and CD3 molecules together comprise the TCR complex.

The term “CD3 antibody” or “anti-CD3 antibody” as used herein refers to an antibody which binds specifically to the antigen CD3, in particular human CD3ε (epsilon).

The term “human CD20” or “CD20” refers to human CD20 (UniProtKB/Swiss-Prot No P11836, SEQ ID NO: 29) and includes any variants, isoforms, and species homologs of CD20 which are naturally expressed by cells, including tumor cells, or are expressed on cells transfected with the CD20 gene or cDNA. Species homologs include rhesus monkey CD20 (macaca mulatta; UniProtKB/Swiss-Prot No H9YXP1) and cynomolgus monkey CD20 (Macaca fascicularis; UniProtKB No G7PQ03).

The term “CD20 antibody” or “anti-CD20 antibody” as used herein refers to an antibody which binds specifically to the antigen CD20, in particular to human CD20.

The term “CD3×CD20 antibody”, “anti-CD3×CD20 antibody”, “CD20×CD3 antibody” or “anti-CD20×CD3 antibody” as used herein refers to a bispecific antibody which comprises two different antigen-binding regions, one of which binds specifically to the antigen CD20 and one of which binds specifically to CD3.

The term “DuoBody-CD3×CD20” as used herein refers to an IgG1 bispecific CD3×CD20 antibody comprising a first heavy and light chain pair as defined in SEQ ID NO: 24 and SEQ ID NO: 25, respectively, and comprising a second heavy and light chain pair as defined in SEQ ID NO: 26 and SEQ ID NO: 27. The first heavy and light chain pair comprises a region which binds to human CD3ε (epsilon), the second heavy and light chain pair comprises a region which binds to human CD20. The first binding region comprises the VH and VL sequences as defined by SEQ ID NOs: 6 and 7, and the second binding region comprises the VH and VL sequences as defined by SEQ ID NOs: 13 and 14. This bispecific antibody can be prepared as described in WO 2016/110576.

Antibodies comprising functional variants of the heavy chain, light chains, VL regions, VH regions, or one or more CDRs of the antibodies of the examples as also provided herein. A functional variant of a heavy chain, a light chain, VL, VH, or CDRs used in the context of an antibody still allows the antibody to retain at least a substantial proportion (at least about 90%, 95% or more) of functional features of the “reference” and/or “parent” antibody, including affinity and/or the specificity/selectivity for particular epitopes of CD20 and/or CD3, Fc inertness and PK parameters such as half-life, Tmax, Cmax. Such functional variants typically retain significant sequence identity to the parent antibody and/or have substantially similar length of heavy and light chains. The percent identity between two sequences is a function of the number of identical positions shared by the sequences (i.e., % homology= # of identical positions/total # of positions×100), taking into account the number of gaps, and the length of each gap, which need to be introduced for optimal alignment of the two sequences. The percent identity between two nucleotide or amino acid sequences may e.g., be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J Mol Biol 1970; 48:444-453 algorithm. Exemplary variants include those which differ from heavy and/or light chains, VH and/or VL, and/or CDR regions of the parent antibody sequences mainly by conservative substitutions; e.g., 10, such as 9, 8, 7, 6, 5, 4, 3, 2 or 1 of the substitutions in the variant may be conservative amino acid residue replacements.

Conservative substitutions may be defined by substitutions within the classes of amino acids reflected in the following table:

TABLE 1
Amino acid residue classes for conservative substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Unless otherwise indicated, the following nomenclature is used to describe a mutation: i) substitution of an amino acid in a given position is written as, e.g., K409R which means a substitution of a Lysine in position 409 with an Arginine; and ii) for specific variants the specific three or one letter codes are used, including the codes Xaa and X to indicate any amino acid residue. Thus, the substitution of Lysine with Arginine in position 409 is designated as: K409R, and the substitution of Lysine with any amino acid residue in position 409 is designated as K409X. In case of deletion of Lysine in position 409 it is indicated by K409*.

The term “humanized antibody” as used herein refers to a genetically engineered non-human antibody, which contains human antibody constant domains and non-human variable domains modified to contain a high level of sequence homology to human variable domains. This can be achieved by grafting of the six non-human antibody CDRs, which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e., the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. The VH and VL of the CD3 arm that is used herein in DuoBody-CD3×CD20 represents a humanized antigen-binding region. Optionally, additional amino acid modifications, which are not necessarily back-mutations, may be applied to obtain a humanized antibody with preferred characteristics, such as affinity and biochemical properties.

The term “human antibody” as used herein refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The VH and VL of the CD20 arm that is used in DuoBody-CD3×CD20 represents a human antigen-binding region. Human monoclonal antibodies of the invention can be produced by a variety of techniques, including conventional monoclonal antibody methodology, e.g., the standard somatic cell hybridization technique of Kohler and Milstein, Nature 256:495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known. Human monoclonal antibodies can thus be generated using, e.g., transgenic or transchromosomal mice or rats carrying parts of the human immune system rather than the mouse or rat system. Accordingly, in one embodiment, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogenous animal antibody machinery (see, e.g., Mendez et al. Nat Genet 1997; 15:146-56). The VH and VL regions of the CD20 arm that is used in DuoBody-CD3×CD20 represents a human antigen-binding region.

The term “biosimilar” (e.g., of an approved reference product/biological drug) as used herein refers to a biologic product that is similar to the reference product based on data from (a) analytical studies demonstrating that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product). In some embodiments, the biosimilar biological product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. A biosimilar can be, e.g., a presently known antibody having the same primary amino acid sequence as a marketed antibody, but may be made in different cell types or by different production, purification, or formulation methods.

The term “reducing conditions” or “reducing environment” as used herein refers to a condition or an environment in which a substrate, here a cysteine residue in the hinge region of an antibody, is more likely to become reduced than oxidized.

The term “recombinant host cell” (or simply “host cell”) as used herein is intended to refer to a cell into which an expression vector has been introduced, e.g., an expression vector encoding an antibody described herein. Recombinant host cells include, for example, transfectomas, such as CHO, CHO-S, HEK, HEK293, HEK-293F, Expi293F, PER.C6 or NS0 cells, and lymphocytic cells.

The term “indolent lymphoma” as used herein refers to a group of slow-growing non-Hodgkin lymphomas (NHLs) including follicular lymphoma grades 1-3A, cutaneous T-cell lymphoma, marginal zone lymphoma, chronic lymphocytic leukemia and Waldenstrom macroglobulinemia. Indolent lymphoma accounts for about 41 percent of all non-Hodgkin lymphoma cases in North America and Northern Europe. As a generalization, indolent lymphomas respond to treatment and are kept under control (in remission) with long-term survival of many years but are not cured.

The term “aggressive lymphoma” as used herein refers to a group of non-Hodgkin lymphomas (NHLs), which usually require intensive treatments. Aggressive lymphomas include large B-cell lymphoma (LBCL), such as Diffuse large B-cell lymphoma (DLBCL), high-grade B-cell lymphoma (HGBCL), primary mediastinal large B-cell lymphoma (PMBCL), follicular lymphoma (FL) grade 3B, mantle cell lymphoma (MCL). Diffuse large B-cell lymphoma (DLBCL) is the most common type of NHL accounting for approximately 30% to 40% of all NHL diagnoses,

“Follicular lymphoma” (FL) as used herein Follicular lymphoma (FL) is a lymphoproliferative disorder generally associated with an indolent course (Freedman, et al., American Journal of Hematology 95 (3): 316-317, 2020). FL originates from follicular center B cells, and about 85% of cases show t (14;18) (q32;q21) chromosomal translocation, which causes the overexpression of the anti-apoptotic protein Bcl-2. FL is characterized by diffuse lymphadenopathy, bone marrow involvement, and splenomegaly. Involvement of non-lymphatic areas is less common. FL grades 1-3A is the most prevalent form of indolent lymphoma, accounting for 70% of indolent cases and 20-30% of all non-Hodgkin lymphoma cases, with a yearly incidence of 1.6 to 3.1 per 100,000. It is most frequently diagnosed among people in their 50s and 60s, and is more common among white populations than black or Asian populations.

“Cutaneous T-cell lymphoma” (CTCL) as used herein is a form of lymphoma derived from T cells. Mycosis fungoides, which attacks the skin, is the most common form of CTCL (Bagherini et al. F1000Research 5:1882, 2016). When cancer cells infiltrate and accumulate in the blood, it is known as Sézary syndrome.

“Marginal zone lymphoma” (MZL) refers to a heterogeneous group of indolent B cell lymphomas that arise from the marginal zone of lymphoid tissues. It accounts for 5-10% of all NHL cases, with an annual incidence of 0.4 to 1.0 per 100,000 in Western countries. The World Health Organization categorizes MZL into three subtypes: nodal, extranodal, and splenic. Nodal MZL occurs within the lymph nodes. Extranodal MZL occurs in areas outside the lymph nodes, with the stomach being the most common site. Splenic MZL develops in the spleen and may spread to the blood.

“Chronic lymphocytic leukemia” (CLL) and “small cell lymphocytic lymphoma” (SLL) refer to different manifestations of the same disease. When the abnormal lymphocytes are located mostly in the lymph nodes, it is referred to as SLL; when the abnormal lymphocytes are mostly in the blood and bone marrow, it is called CLL. CLL is the most common leukemia in Western countries. The median age at diagnosis is 72 years.

The terms “DLBCL” and “HGBCL” as defined herein refers to B-NHL classified as either diffuse large B-cell lymphoma (DLBCL) or high-grade B-cell lymphoma (HGBCL), in accordance with the WHO classification as defined in Swerdlow S H, Campo E, Harris N L, et al. WHO Classification of Tumours of Haematopoietic and Lymphoid Tissues (ed. 4th). Lyon, France: IARC Press (2008) and Swerdlow S H, Campo E, Harris N L, et al. WHO Classification of Tumours of Haematopoietic and Lymphoid Tissues (Revised ed. 4th). Lyon, France: IARC Press (2017), which are incorporated herein by reference.

“MCL”, i.e. Mantle cell lymphoma, comprises B-cell lymphoma with chromosomal translocation t (11; 14) leading to expression of cyclin D1, also including CD5+. MCL as defined herein includes B-NHL classified as MCL in accordance with the WHO classification as defined in Swerdlow S H, Campo E, Harris N L, et al. WHO Classification of Tumours of Haematopoietic and Lymphoid Tissues (ed. 4th). Lyon, France: IARC Press (2008) and Swerdlow S H, Campo E, Harris N L, et al. WHO Classification of Tumours of Haematopoietic and Lymphoid Tissues (Revised ed. 4th). Lyon, France: IARC Press (2017), which are incorporated herein by reference.

The Lugano modification of the Ann Arbor system is used to stage lymphoma as follows:
Stage I: The lymphoma is in one lymph node or one group of lymph nodes; or, in rare cases, in one organ of the lymphatic system such as Waldeyer's ring, the thymus, or the spleen; or in one site outside the lymphatic system (IE).
Stage II: The lymphoma is in two or more groups of lymph nodes; or in one nearby area outside the lymphatic system, with or without involvement of other lymph nodes (IIE). In either case, the lymphoma sites are on the same side of the diaphragm. In Stage II, “bulky disease” means tumor mass larger than a certain size; the threshold depends on the type of lymphoma.
Stage III: The lymphoma is on both sides of the diaphragm, either in lymph nodes both above and below the diaphragm, or in lymph nodes above the diaphragm and in the spleen.
Stage IV: The lymphoma is in one or more organs beyond the lymphatic system, such as the liver, lungs, bone marrow, or cerebrospinal fluid.

A patient's stage may be determined through blood tests, bone marrow biopsy, chest X-rays, computed tomography (CT) scans, positron emission tomography (PET) scans, and magnetic resonance imaging (MRI).

The term “relapsed lymphoma” as used herein refers to lymphoma which progressed after achieving partial response (PR) or complete response (CR) to prior treatment with an anti-neoplastic therapy.

The term “refractory lymphoma” as used herein refers to lymphoma which was treated with at least one prior anti-neoplastic therapy but failed to achieve at least a partial response to the therapy.

The term “autologous stem cell transplant” or “ASCT” as used herein refers to stem cells that are collected from an individual and given back to that the individual.

The term “treatment” refers to the administration of an effective amount of a therapeutically active antibody described herein for the purpose of easing, ameliorating, arresting or eradicating (curing) symptoms or disease states such as DLBCL. Treatment may result in a complete response (CR), partial response (PR), or stable disease (SD), for example, as defined by Lugano criteria and/or LYRIC. Treatment may be continued, for example, until disease progression or unacceptable toxicity.

The term “administering” or “administration” as used herein refers to the physical introduction of a composition (or formulation) comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a therapeutic agent described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods. In the methods described herein, the bispecific antibody (e.g., epcoritamab) is administered subcutaneously. Other agents used in combination with the bispecific antibody, such as GemOx, cytokine release syndrome prophylaxis, and/or tumor lysis syndrome (TLS) prophylaxis, may be administered via other routes, such as intravenously or orally.

The term “effective amount” or “therapeutically effective amount” refers to an amount effective, at dosages and for periods of time necessary, to achieve a desired therapeutic result. For example, dosages as defined herein for the bispecific antibody (e.g., epcoritamab), i.e., 24 mg or 48 mg, administered subcutaneously can be defined as such an “effective amount” or “therapeutically effective amount”. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects. In some embodiments, patients treated with the methods described herein will show an improvement in ECOG performance status. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or disorder (e.g., cytokine release syndrome) or of suffering a recurrence of disease, inhibits the development or recurrence of the disease.

The term “inhibits growth” of a tumor as used herein includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%.

The term “subject” as used herein refers to a human patient, for example, a human patient with B-NHL, such as FL or LBCL, including DLBCL. The terms “subject” and “patient” are used interchangeably herein.

The term “buffer” as used herein denotes a pharmaceutically acceptable buffer. The term “buffer” encompasses those agents which maintain the pH value of a solution, e.g., in an acceptable range and includes, but is not limited to, acetate, histidine, TRIS® (tris (hydroxymethyl) aminomethane), citrate, succinate, glycolate and the like. Generally, the “buffer” as used herein has a pKa and buffering capacity suitable for the pH range of about 5 to about 6, preferably of about 5.5.

“Disease progression” or “PD” as used herein refers to a situation in which one or more indices of lymphoma show that the disease is advancing despite treatment. In one embodiment, disease progression is defined based on the Lugano Response Criteria for Malignant Lymphoma (“Lugano criteria”) and/or Lymphoma Response to Immunomodulatory Therapy Criteria (LYRIC). Details regarding the Lugano criteria/classification system, including definitions for complete response (CR), partial response (PR), no response/stable disease (NR/SD), and progressive disease (PD) are provided in Cheson et al. J Clin Oncol 2014; 32:3059-68, the contents of which are incorporated by reference herein (see, in particular, Table 3 in Cheson et al., 2014). Details regarding LYRIC are provided in Table 7.

A “surfactant” as used herein is a compound that is typically used in pharmaceutical formulations to prevent drug adsorption to surfaces and or aggregation. Furthermore, surfactants lower the surface tension (or interfacial tension) between two liquids or between a liquid and a solid. For example, an exemplary surfactant can significantly lower the surface tension when present at very low concentrations (e.g., 5% w/v or less, such as 3% w/v or less, such as 1% w/v or less such as 0.4% w/v or less, such as below 0.1% w/v or less, such as 0.04% w/v). Surfactants are amphiphilic, which means they are usually composed of both hydrophilic and hydrophobic or lipophilic groups, thus being capable of forming micelles or similar self-assembled structures in aqueous solutions. Known surfactants for pharmaceutical use include glycerol monooleate, benzethonium chloride, sodium docusate, phospholipids, polyethylene alkyl ethers, sodium lauryl sulfate and tricaprylin (anionic surfactants); benzalkonium chloride, citrimide, cetylpyridinium chloride and phospholipids (cationic surfactants); and alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl hydroxystearate, polyoxylglycerides, polysorbates such as polysorbate 20 or polysorbate 80, propylene glycol dilaurate, propylene glycol monolaurate, sorbitan esters sucrose palmitate, sucrose stearate, tricaprylin and TPGS (Nonionic and zwitterionic surfactants).

A “diluent” as used herein is one which is pharmaceutically acceptable (safe and non-toxic for administration to a human) and is useful for the preparation of dilutions of the pharmaceutical composition or pharmaceutical formulation (the terms “composition” and “formulation” are used interchangeably herein). Preferably, such dilutions of the composition dilute only the antibody concentration but not the buffer and stabilizer. Accordingly, in one embodiment, the diluent contains the same concentrations of the buffer and stabilizer as is present in the pharmaceutical composition of the invention. Further exemplary diluents include sterile water, bacteriostatic water for injection (BWFI), a pH buffered solution which is preferably an acetate buffer, sterile saline solution such as water for injection, Ringer's solution or dextrose solution. In one embodiment the diluent comprises or consists essentially of acetate buffer and sorbitol.

As used herein, the term “about” refers to a value that is no more than 10% above and no more than 10% below a specified value.

In one aspect, the invention provides a method of treating indolent lymphoma in a human subject, the method comprising administering to the subject a bispecific antibody, wherein the bispecific antibody comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14,
wherein the bispecific antibody is administered in a 28 day cycle comprising administering a priming dose on day 1 of about 0.05 mg to about 0.35 mg, a first intermediate dose on about day 8 of about 0.6 mg to 5 mg, a second intermediate dose on about day 15 of about 1 mg to about 10 mg, followed by one weekly dose of between about 20-100 mg.

Alternatively, the bispecific antibody is administered in a 35 day cycle comprising administering a priming dose on day 1 of about 0.05 mg to about 0.35 mg, a first intermediate dose on about day 8 of about 0.6 mg to 5 mg, a second intermediate dose on about day 15 of about 1 mg to about 10 mg, followed by two weekly doses of between about 20-100 mg.

In some embodiments, the priming dose is about 0.16 mg.

In some embodiments, the first intermediate dose is about 0.6-1.2 mg, such as about 0.8 mg.

In some embodiments, the second intermediate dose is about 3-6 mg, such as about 3 mg or such as about 6 mg.

In some embodiments, the weekly dose is between about 20 to 60 mg, such as 24 mg or 48 mg.

In some embodiments, the weekly administration is performed at least 4 times.

Additional embodiments provide a dosing regimen, wherein after the weekly administration, the bispecific antibody is administered once every two weeks. In particular, the biweekly administration of the bispecific antibody may be performed at least six (6) times.

Further, dosing regimens are provided herein, wherein after the biweekly administration, the bispecific antibody is administered once every four weeks.

Preferably, the bispecific antibody is administered subcutaneously.

In some embodiments, the method comprises:
(a) a first cycle of 28-days wherein
   (i) the priming dose of the bispecific antibody is administered on Day 1;
   (ii) the first intermediate dose of the bispecific antibody is administered on Day 8;
   (iii) the second intermediate dose of the bispecific antibody is administered on Day 15;
   (iv) a dose of 24 mg of the bispecific antibody is administered on Day 22; and
(b) cycles 2-3 each comprising 28 days wherein a dose of 24 mg of the bispecific antibody is administered on Days 1, 8, 15 and 22;
(c) cycles 4-9 each comprising 28 days wherein a dose of 24 mg of the bispecific antibody is administered on Days 1 and 15; and
(d) further subsequent 28 day cycles, wherein a dose of 24 mg of the bispecific antibody is administered on Day 1.

In some embodiments, the method comprises:
(a) a first cycle of 35-days wherein
   (i) the priming dose of the bispecific antibody is administered on Day 1;
   (ii) the first intermediate dose of the bispecific antibody is administered on Day 8;
   (iii) the second intermediate dose of the bispecific antibody is administered on Day 15;
   (iv) a dose of 24 mg of the bispecific antibody is administered on Days 22 and 28; and
(b) cycles 2-3 each comprising 28 days wherein a dose of 24 mg of the bispecific antibody is administered on Days 1, 8, 15 and 22;
(c) cycles 4-9 each comprising 28 days wherein a dose of 24 mg of the bispecific antibody is administered on Days 1 and 15; and
(d) further subsequent 28 day cycles, wherein a dose of 24 mg of the bispecific antibody is administered on Day 1.

In some embodiments the method comprises:
(a) a first cycle of 28-days wherein
   (i) the priming dose of the bispecific antibody is administered on Day 1;
   (ii) the first intermediate dose of the bispecific antibody is administered on Day 8;
   (iii) the second intermediate dose of the bispecific antibody is administered on Day 15;
   (iv) a dose of 48 mg of the bispecific antibody is administered on Day 22; and
(b) cycles 2-3 each comprising 28 days wherein a dose of 48 mg of the bispecific antibody is administered on Days 1, 8, 15 and 22;
(c) cycles 4-9 each comprising 28 days wherein a dose of 48 mg of the bispecific antibody is administered on Days 1 and 15; and
(d) further subsequent 28 day cycles, wherein a dose of 48 mg of the bispecific antibody is administered on Day 1.

In some embodiments the method comprises:
(a) a first cycle of 35-days wherein
   (i) the priming dose of the bispecific antibody is administered on Day 1;
   (ii) the first intermediate dose of the bispecific antibody is administered on Day 8;
   (iii) the second intermediate dose of the bispecific antibody is administered on Day 15;
   (iv) a dose of 48 mg of the bispecific antibody is administered on Days 22 and 28; and
(b) cycles 2-3 each comprising 28 days wherein a dose of 48 mg of the bispecific antibody is administered on Days 1, 8, 15 and 22;
(c) cycles 4-9 each comprising 28 days wherein a dose of 48 mg of the bispecific antibody is administered on Days 1 and 15; and

(d) further subsequent 28 day cycles, wherein a dose of 48 mg of the bispecific antibody is administered on Day 1.

In some embodiments, the method comprises:
(a) a first cycle of 28-days wherein
   (i) a priming dose of 0.16 mg of the bispecific antibody is administered on Day 1;
   (ii) a first intermediate dose 0.8 mg of the bispecific antibody is administered on Day 8;
   (iii) a second intermediate dose of 3 mg of the bispecific antibody is administered on Day 15;
   (iv) a dose of 48 mg of the bispecific antibody is administered on Day 22; and
(b) cycles 2-3 each comprising 28 days wherein a dose of 48 mg of the bispecific antibody is administered on Days 1, 8, 15 and 22;
(c) cycles 4-9 each comprising 28 days wherein a dose of 48 mg of the bispecific antibody is administered on Days 1 and 15; and
(d) further subsequent 28 day cycles, wherein a dose of 48 mg of the bispecific antibody is administered on Day 1.

In some embodiments, the method comprises:
(a) a first cycle of 35-days wherein
   (i) a priming dose of 0.16 mg of the bispecific antibody is administered on Day 1;
   (ii) a first intermediate dose 0.8 mg of the bispecific antibody is administered on Day 8;
   (iii) a second intermediate dose of 3 mg of the bispecific antibody is administered on Day 15;
   (iv) a dose of 48 mg of the bispecific antibody is administered on Days 22 and 28; and
(b) cycles 2-3 each comprising 28 days wherein a dose of 48 mg of the bispecific antibody is administered on Days 1, 8, 15 and 22;
(c) cycles 4-9 each comprising 28 days wherein a dose of 48 mg of the bispecific antibody is administered on Days 1 and 15; and
(d) further subsequent 28 day cycles, wherein a dose of 48 mg of the bispecific antibody is administered on Day 1.

In some embodiments, the method comprises:
(a) a first cycle of 35-days wherein
   (i) a priming dose of 0.16 mg of the bispecific antibody is administered on Day 1;
   (ii) a first intermediate dose 0.8 mg of the bispecific antibody is administered on Day 8;
   (iii) a second intermediate dose of 6 mg of the bispecific antibody is administered on Day 15;
   (iv) a dose of 48 mg of the bispecific antibody is administered on Days 22 and 28; and
(b) cycles 2-3 each comprising 28 days wherein a dose of 48 mg of the bispecific antibody is administered on Days 1, 8, 15 and 22;
(c) cycles 4-9 each comprising 28 days wherein a dose of 48 mg of the bispecific antibody is administered on Days 1 and 15; and
(d) further subsequent 28 day cycles, wherein a dose of 48 mg of the bispecific antibody is administered on Day 1.

Briefly, B-NHLs are typically divided into indolent (slow-growing) and aggressive subtypes. Aggressive B-NHLs have high Ki67 expression, whereas indolent B-NHLs have relatively low Ki67 expression. As a generalization, indolent lymphomas respond to treatment and are kept under control (in remission) with long-term survival of many years but are not cured. Aggressive lymphomas usually require intensive treatments, with some having a good prospect for a permanent cure. Aggressive B-NHL includes: Diffuse large B-cell lymphoma (DLBCL), high-grade B-cell lymphoma (HGBCL), primary mediastinal large B-cell lymphoma (PMBCL), follicular lymphoma (FL) grade 3B, mantle cell lymphoma (MCL). Indolent B-NHL includes FL grades 1-3A, marginal-zone lymphoma (MZL) and small lymphocytic lymphoma (SLL). Diffuse large B-cell lymphoma (DLBCL) is the most common type of NHL accounting for approximately 30% to 40% of all NHL diagnoses, followed by FL (20% to 25% of all NHL diagnoses). The majority of the B-cell lymphomas express B-cell markers, such as CD19, CD20, CD22, and CD79b. The biologic heterogeneity of B-cell malignancies is reflected in the clinical course and outcome of individual diseases. Indolent diseases such as FL G1-3A, MZL, and SLL evolve slowly, with a median survival of 8 to 10 years. In contrast, more aggressive diseases such as DLBCL/HGBCL, if left untreated, have a median survival of 6 months. The median age at diagnosis for most patients with lymphoma is approximately 60 to 65 years (WHO, 2008).

### Aggressive Lymphoma Treatment Regimens

Provided herein are additional methods of treating aggressive lymphoma, such as Large B-Cell lymphoma (LBCL) in a human subject using a bispecific antibody which binds to CD3 and CD20 (“anti-CD3×CD20 antibody”), e.g., an isolated anti-CD3×CD20 antibody such as epcoritamab which binds to human CD3 and human CD20 in a treatment regimen comprising administration of a first priming dose of 0.5 to 0.35 mg, a first intermediate dose of 0.6 to 5 mg, a second intermediate dose of 1 to 10 mg of the anti-CD3×CD20 antibody, followed by at least one dose of 20-100 mg of the bispecific antibody.

In some embodiments, the priming dose is about 0.16 mg.

In some embodiments, the first intermediate dose is about 0.6-1.2 mg, such as about 0.8 mg.

In some embodiments, the second intermediate dose is about 3-6 mg. In particular, the second intermediate dose may be about 3 mg. Alternatively, the second intermediate dose may be about 6 mg.

In particular embodiments, the bispecific antibody is administered in a first cycle, such as a 21 day cycle, comprising administering a first priming dose on day 1 of about 0.05 mg to about 0.35 mg, a first intermediate dose on about day 8 of about 0.6 mg to 5 mg, a second intermediate dose on about day 15 of about 1 mg to about 10 mg.

Following the first cycle the bispecific antibody may be administered in
(a) cycles 2-4 each comprising 21 days wherein a dose of about 24 or about 48 mg of the bispecific antibody is administered weekly, such as on about Days 1, 8, 15;
(b) cycles 5-6 each comprising 21 days wherein a dose of about 24 or about 48 mg of the bispecific antibody is administered twice on about Days 1 and 15; and
(c) further subsequent 28 day cycles, wherein a dose of about 24 or about 48 mg of the bispecific antibody is administered once, such as on about Day 1.

The method according to this embodiment, may further comprise administration of therapeutically effective amounts of rituximab, cyclophosphamide, doxorubicin, and vincristine. In particular, the method may be for treatment of DLBCL, such as previously untreated DLBCL.

In an alternative embodiment is provided a method, wherein following the first cycle, the bispecific antibody is administered in
(a) cycles 2-4 each comprising 21 days wherein a dose of about 24 or about 48 mg of the bispecific antibody is administered weekly, such as on about Days 1, 8, 15;
(b) cycles 5-9 each comprising 21 days wherein a dose of about 24 or about 48 mg of the bispecific antibody is administered twice, such as on about Days 1 and 15; and
(c) further subsequent cycles each comprising 28 day, wherein a dose of about 24 or about 48 mg of the bispecific antibody is administered once, such as on about Day 1.

The method according to this embodiment, may further comprise administration of therapeutically effective amounts of rituximab, dexamethasone, cytarabine, and oxaliplatin/carboplatin. In particular, the method may be for treatment of DLBCL, such as relapsed/refractory DLBCL, in particular DLBCL eligible for autologous stem cell transplantation (ASCT).

In a further alternative embodiment is provided a method, wherein following the first cycle, the bispecific antibody is administered in
(a) cycle 2 comprising 21 days wherein a dose of about 24 or about 48 mg of the bispecific antibody is administered weekly, such as on about Days 1, 8, 15;
(b) cycles 3-6 each comprising 21 days, wherein a dose of about 24 or about 48 mg of the bispecific antibody is administered twice, such as on about Day 1; and
(c) further subsequent cycles each comprising 28 days, wherein a dose of about 24 or about 48 mg of the bispecific antibody is administered once, such as on about Day 1.

The method according to this embodiment, may further comprise administration of therapeutically effective amounts of rituximab, cyclophosphamide, doxorubicin, and vincristine. In particular, the method may be for treatment of DLBCL, such as in subjects with previously untreated DLBCL who are ineligible to receive full therapeutic dose of anthracycline.

### Indolent Lymphoma Treatment Regimens

Provided herein are methods of treating FL in a human subject using a bispecific antibody which binds to CD3 and CD20 (“anti-CD3×CD20 antibody”), e.g., an isolated anti-CD3×CD20 antibody such as epcoritamab which binds to human CD3 and human CD20 in a treatment regimen comprising administration of a first priming dose of 0.5 to 0.35 mg, a first intermediate dose of 0.6 to 5 mg, a second intermediate dose of 1 to 10 mg of the anti-CD3×CD20 antibody, followed by at least two weekly doses of 20-100 mg of the bispecific antibody.

Accordingly, in one aspect, provided herein is a method of treating indolent lymphoma (e.g., FL) in a human subject, the method comprising administering a bispecific antibody wherein the bispecific antibody comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
   wherein the bispecific antibody is administered at a priming dose of 0.05 mg to about 0.35 mg, a first intermediate dose of about 0.6 mg to 5 mg, a second intermediate dose of about 1 mg to about 10 mg, followed by a dose of 24 mg or 48 mg.

In some embodiments, the priming dose is about 0.16 mg.

In some embodiments, the first intermediate dose is about 0.6-1.2 mg, such as about 0.8 mg.

In some embodiments, the second intermediate dose is about 3-6 mg. In particular, the second intermediate dose may be about 3 mg. Alternatively, the second intermediate dose may be about 6 mg.

In some embodiments, the bispecific antibody is administered at a dose of (or a dose of about) 24 mg. In some embodiments, the bispecific antibody is administered at a dose of (or a dose of about) 48 mg.

In particular embodiments, the bispecific antibody is administered in a first cycle, such as a 28 day cycle, comprising administering a first priming dose on day 1 of about 0.05 mg to about 0.35 mg, a first intermediate dose on about day 8 of about 0.6 mg to 5 mg, a second intermediate dose on about day 15 of about 1 mg to about 10 mg and a dose of about 24 or about 48 mg on about day 22.

Following the first cycle the bispecific antibody may be administered in
(b) cycle 2 comprising 28 days wherein a dose of about 24 or about 48 mg of the bispecific antibody is administered weekly, such as on about Days 1, 8, 15, 22;
(c) cycles 3-26 each comprising 28 days wherein a dose of about 24 or about 48 mg of the bispecific antibody is administered once, such as on about Day 1.

The method according to this embodiment, may further comprise administration of therapeutically effective amounts of rituximab and lenalidomide. The method may in particular be for treatment of relapsed/refractory FL. Alternatively, the method may be for treatment of previously untreated, advanced FL.

In an alternative embodiment is provided a method, wherein following the first cycle, the bispecific antibody is administered at a dose of about 24 or about 48 mg once every eight week.

This method is primarily intended as maintenance therapy in subjects with FL, that are in complete response or partial response following first- or second line treatment.

In some embodiments, the bispecific antibody is a full length antibody. In other embodiments, the bispecific antibody is an antibody with an inert Fc region. In yet other embodiments, the bispecific antibody is a full length antibody with an inert Fc region.

With regard to the dose of (or dose of about) 24 mg or 48 mg of the bispecific antibody that is to be administered, or any other specified dose, it is understood that this amount refers to the amount of a bispecific antibody representing a full-length antibody, such as epcoritamab as defined in the Examples section. Hence, one may refer to administering a dose of a bispecific antibody of 24 mg as administering a dose of a bispecific antibody described herein, wherein the dose corresponds to a dose of 24 mg of epcoritamab. One of ordinary skill in the art can readily determine the amount of antibody to be administered when, for example, the antibody used differs substantially in molecular weight from the molecular weight of a full-length antibody such as epcoritamab. For instance, the amount of antibody can be calculated by dividing the molecular weight of the antibody by the weight of a full-length antibody such as epcoritamab and multiplying the outcome thereof with the specified dose as described herein. As long as the bispecific antibody (e.g., a functional variant of DuoBody CD3×CD20) has highly similar features as DuoBody CD3×CD20, with regard to plasma half-life, Fc inertness, and/or binding characteristics for CD3 and CD20, i.e., with regard to CDRs and epitope binding features, such antibodies are suitable for use in the methods provided herein at a dose described for a full-length antibody such as epcoritamab.

In some embodiments, the dose of bispecific antibody is administered once a week (weekly administration) in 28-day cycles. In one embodiment, the weekly dose of 24 mg or 48 mg is administered for 2.5 28-day cycles (i.e., 10 times; on days 15 and 22 of cycle 1, and days 1, 8, 15, and 22 of cycles 2-3). In other embodiments, after the weekly administration, one may reduce the interval of administration to once every two weeks (biweekly administration). In one embodiment, the biweekly administration is performed for six 28-day cycles (i.e., 12 times). In some embodiments, after the biweekly administration, one may reduce the interval of administration to once every four weeks. In one embodiment, the administration once every four weeks may be performed for an extended period, for example, for at least 1 cycle, at least 2 cycles, at least 3 cycles, at least 4 cycles, at least 5 cycles, at least 6 cycles, at least 7 cycles, at least 8 cycles, at least 9 cycles, at least 10 cycles, at least 11 cycles, at least 12 cycles, at least 13 cycles, at least 14 cycles, at least 15 cycles, at least 16 cycles, at least 17 cycles, such as between 1-20 cycles, 1-19 cycles, 1-18 cycles, 1-17 cycles, 1-16 cycles, 1-15 cycles, 1-14 cycles, 1-13 cycles, 1-12 cycles, 1-10 cycles, 1-5 cycles, 5-20 cycles, 5-15 cycles, or 5-10 cycles of the 28-day cycles. In some embodiments, epcoritamab is administered as monotherapy (i.e., without GemOx) from cycle 10 of the 28-day cycles. In some embodiments, epcoritamab is administered as monotherapy from cycle 10 to cycle 26 of the 28-day cycles. In some embodiments, epcoritamab is administered as monotherapy from cycle 7 of the 28-day cycles until disease progression (e.g., as defined by the Lugano criteria or LYRIC) or unacceptable toxicity.

In one embodiment, the weekly dose of the bispecific antibody is administered in 28-day cycles on cycles 1-3 (which may include priming and intermediate doses, as described below), the biweekly dose of the bispecific antibody is administered on cycles 4-9, and the dose once every four weeks is administered from cycle 10 onwards, for example, on cycles 10-15, cycles 10-20, cycles 10-25, cycles 10-30, or more cycles, e.g., until disease progression or unacceptable toxicity is observed in the subject. In some embodiments, the dose once every four weeks is administered on cycles 10-26.

It is understood that the doses referred to herein may also be referred to as a full or a flat dose in the scenarios above wherein, e.g., the weekly dose, biweekly dose, and/or the dose every four weeks is administered is at the same level. Accordingly, when a dose of 48 mg is selected, preferably, at each weekly administration, at each biweekly administration, and each administration every four weeks, the same dose of 48 mg is administered. Prior to administering the dose, a priming or a priming and subsequent first and second intermediate doses are be administered. This may be advantageous as it may help mitigate cytokine release syndrome (CRS) risk and severity, a side-effect that can occur during treatment with the bispecific anti-CD3×CD20 antibody described herein. Such priming, or priming and intermediate doses, are at a lower dose as compared with the flat or full dose.

Accordingly, in some embodiments, prior to administering the weekly dose of 24 mg or 48 mg, a priming dose of the bispecific antibody may be administered. In one embodiment, the priming dose is administered two weeks prior to administering the first weekly dose of 24 mg or 48 mg in cycle 1. In one embodiment, the priming dose is 0.16 mg (or about 0.16 mg) of the full-length bispecific antibody.

In some embodiments, after administering the priming dose and prior to administering the weekly dose of 24 mg or 48 mg, a first intermediate dose of said bispecific antibody is administered. In one embodiment, the priming dose is administered one week before the first intermediate dose (i.e., on day 1 of cycle 1), and the first intermediate dose is administered one week before the second intermediate dose (i.e., on day 8 of cycle 1) and the second intermediate dose is administered before the first weekly dose of 24 mg or 48 mg (i.e., on day 15 of cycle 1). In one embodiment, the priming dose is 0.16 mg of the full-length bispecific antibody. In one embodiment, the first intermediate dose is 800 μg (0.8 mg) or about 800 μg (0.8 mg) of the full-length bispecific antibody. In one embodiment, the second intermediate dose is 3 mg of the full-length bispecific antibody. In one embodiment, the second intermediate dose is 6 mg of the full-length bispecific antibody.

In some embodiments, the subject is administered premedication and/or prophylaxis for CRS prior to administration of the bispecific antibody.

In one embodiment, the subject undergoing the treatment with the methods described herein has documented indolent lymphoma according to the WHO 2016 classification (Swerdlow S H, Campo E, Harris N L, et al. WHO Classification of Tumours of Haematopoietic and Lymphoid Tissues (Revised ed. 4th). Lyon, France: IARC Press (2017), the contents of which are herein incorporated by reference). In some embodiments, the subject has FL. In some embodiments, the subject has follicular lymphoma Grade 3B. In a further embodiment, the subject has relapsed after or is refractory to at least one prior therapy. In yet a further embodiment, the subject has failed prior autologous HSCT. In yet a further embodiment, the subject is ineligible to receive autologous HSCT, for example, due to age, performance status, comorbidities, and/or insufficient response to prior treatment.

In some embodiments, the subject has an Eastern Cooperative Oncology Group (ECOG) performance status (ECOG PS) of 0, 1, or 2. Information regarding ECOG PS scores can be found in, e.g., Oken et al, Am J Clin Oncol 1982 December; 5 (6): 649-55).

In some embodiments, the subject has measurable disease as defined as (a) ≥1 measurable nodal lesion (long axis >1.5 cm and short axis >1.0 cm) or ≥1 measurable extranodal lesion (long axis >1 cm) on CT or MRI.

In some embodiments, the subject has acceptable organ function as defined as: (a) ANC ≥1.0×10⁹/L, (b) platelet count >75×10⁹/L, or ≥50×10⁹/L if bone marrow infiltration or splenomegaly, (c) ALT level ≤2.5 times the ULN, (d) total bilirubin level ≤2×ULN, (e) eGFR >50 mL/min (by Cockcroft-Gault Formula), and (f) PT, INR, and aPTT≤1.5×ULN (unless receiving anticoagulant).

In some embodiments, the subject does not have severe allergic or anaphylactic reactions to anti-CD20 antibody therapy, or the bispecific antibody, or known allergy or intolerance to any component or excipient of the bispecific antibody formulation.

In some embodiments, the subject does not have clinically significant cardiac disease, including (a) myocardial infarction within one year prior to the first dose of the bispecific antibody, or unstable or uncontrolled disease/condition related to or affecting cardiac function (e.g., unstable angina, congestive heart failure, NYHA class III-IV), cardiac arrhythmia (CTCAE Version 4 Grade 2 or higher), or clinically significant ECG abnormalities, and/or (b) 12-lead ECG showing a baseline QTcF >470 msec.

A human subject receiving a treatment described herein may be a patient having one or more of the inclusion criteria set forth in the Examples.

The methods described herein are advantageous for treating indolent lymphoma, such as FL. The treatment is maintained continuously using, e.g., the treatment regimens described herein, until progressive disease develops or unacceptable toxicity occurs.

The response of subjects with indolent lymphoma to treatment using the methods described herein may be assessed according to the Lugano Response Criteria for Malignant Lymphoma (also referred to as “Lugano criteria” herein) and/or Lymphoma Response to Immunomodulatory Therapy Criteria (also referred to as “LYRIC” herein), as described in the Examples. In one embodiment, complete response (CR), partial response (PR), and stable disease (SD) are assessed using the Lugano criteria. In some embodiments, patients showing disease progression, also referred to as progressive disease (PD), according to the Lugano criteria are further evaluated according to LYRIC. Details regarding the Lugano criteria/classification system, including definitions for complete response, partial response, no response/stable disease, and progressive disease are provided in Cheson et al. J Clin Oncol 2014; 32:3059-68 (see, in particular, Table 3 in Cheson et al., 2014). Details regarding LYRIC are provided in Table 7.

In some embodiments, subjects are treated with the methods described herein until they show disease progression (PD), e.g., as defined by Lugano criteria and/or LYRIC. In one embodiment, subjects are treated with the methods described herein until they show disease progression (PD) as defined by both Lugano criteria and LYRIC.

Subjects treated according to the methods described herein preferably experience improvement in at least one sign of indolent lymphoma (e.g., FL). In one embodiment, improvement is measured by a reduction in the quantity and/or size of measurable tumor lesions. In some embodiments, lesions can be measured on CT, PET-CT, or MRI films. In some embodiments, cytology or histology can be used to evaluate responsiveness to a therapy. In some embodiments, bone marrow aspirate and bone marrow biopsy can be used to evaluate response to therapy.

In one embodiment, the subject treated exhibits a complete response (CR), a partial response (PR), or stable disease (SD), as defined by the Lugano criteria or LYRIC (see, e.g., Table 7). In some embodiments, the methods described herein produce at least one therapeutic effect chosen from prolonged survival, such as progression-free survival or overall survival, optionally compared to another therapy or placebo.

In some embodiments, T cell activity (e.g., CD4+ and/or CD8+ T cell activity) is increased in subjects treated with the combination of bispecific antibody, gemcitabine, and oxaliplatin. In some embodiments, CD69, CD25, PD-1, and/or LAMP-1 expression is increased in CD4+ and/or CD8+ T cells from subjects treated with the combination of bispecific antibody, gemcitabine, and oxaliplatin.

In some embodiments, anti-tumor activity (e.g., B cell cytotoxicity) is increased in subjects treated with the combination of bispecific antibody, gemcitabine, and oxaliplatin, e.g., relative to subjects treated with the bispecific antibody alone, or a combination of the bispecific antibody with gemcitabine or oxaliplatin.

Cytokine release syndrome (CRS) can occur when methods are used in human subjects that utilize immune cell- and bispecific antibody-based approaches that function by activation of immune effector cell, such as by engaging CD3 (Lee et al., Biol Blood Marrow Transplant 2019; 25:625-38, which is incorporated herein by reference). Hence, in some embodiments, CRS mitigation is performed together with the methods described herein. As part of CRS mitigation, the selection of a priming dose and first and second intermediate dose sis performed prior to administering the full dose (e.g., 24 or 48 mg), as described herein. CRS can be classified in accordance with standard practice (e.g., as outlined in Lee et al., Biol Blood Marrow Transplant 2019; 25:625-38, which is incorporated herein by reference). CRS may include excessive release of cytokines, for example of proinflammatory cytokines, e.g., IL-6, TNF-alpha, or IL-8, which may result in adverse effects like fever, nausea, vomiting and chills. Thus, despite the unique anti-tumor activity of bispecific antibodies such as epcoritamab, their immunological mode of action may trigger unwanted “side” effects, i.e., the induction of unwanted inflammatory reactions. Hence, patients may be further subjected to a concomitant treatment, prophylaxis, and/or premedication with, e.g., analgesics, antipyretics, and/or anti-inflammatory drugs to mitigate possible CRS symptoms.

Accordingly, in one embodiment, human subjects in the methods described herein are treated with prophylaxis for CRS. In some embodiments, the prophylaxis includes the administration of a corticosteroid. In one embodiment, the prophylaxis is administered on the same day as the bispecific antibody. The prophylaxis can also be administered on the subsequent day as well, more preferably on subsequent days 2, 3, and 4. It is understood that days 2, 3 and 4 when relating to further medication, such as prophylaxis, is relative to the administration of the bispecific antibody which is administered on day 1. For example, when in a cycle the antibody is administered on day 15, and prophylaxis is also administered, the prophylaxis corresponding to days 2, 3 and 4 are days 16, 17, and 18 of the cycle. In some embodiments, the prophylaxis is administered on the day when the bispecific antibody is administered and on subsequent days 2-4. When said prophylaxis is administered on the same day as the bispecific antibody, the prophylaxis is preferably administered 30-120 minutes prior to said administration of the bispecific antibody. An exemplary corticosteroid suitable for use in the methods and uses described herein is dexamethasone. In some embodiments, prednisolone is administered at an intravenous dose of 15 mg, or an equivalent thereof, including an oral dose. Exemplary corticosteroid equivalents of dexamethasone, along with dosage equivalents, which can be used for CRS prophylaxis are shown in Table 4.

Furthermore, in some embodiments, human subjects in the methods described herein are treated with premedication to reduce reactions to injections. In one embodiment, the premedication includes the administration of antihistamines. In some embodiments, the premedication includes the administration of antipyretics. In a further embodiment, the premedication includes systemic administration of antihistamines and antipyretics.

An exemplary antihistamine suitable for use in premedication is diphenhydramine. In one embodiment, diphenhydramine is administered at an intravenous or oral dose 50 mg, or an equivalent thereof. An exemplary antipyretic suitable for use in premedication is acetaminophen. In one embodiment, acetaminophen is administered at an oral dose of 650-1000 mg, or equivalent thereof. In some embodiments, the premedication is administered on the same day as the bispecific antibody, for example, prior to the injection with the bispecific antibody, e.g., 30-120 minutes prior to administration of the bispecific antibody.

Premedication and/or prophylaxis for CRS can be administered at least in the initial phase of the treatment. In some embodiments, premedication and/or prophylaxis is administered during the first four administrations of the bispecific antibody. For example, the prophylaxis can be administered as described herein, during the first 28-day cycle of the bispecific antibody administration. In some embodiments, the premedication is administered during said first cycle.

Usually, risk of reactions during the initial treatment subsides after a few administrations, e.g., after the first four administrations (first cycle). Hence, when the human subject does not experience CRS with the fourth administration, prophylaxis for CRS may be stopped. However, CRS prophylaxis may continue, particularly when the human subject experiences a CRS greater than grade 1. Likewise, premedication may also optionally continue. CRS grading can be performed as described in Tables 5 and 6.

In a further embodiment, in the methods described herein, the prophylaxis for CRS is administered during the second 28-day cycle when the human subject experiences CRS greater than grade 1 after the fourth administration of the bispecific antibody in cycle 1. Furthermore, the prophylaxis can be continued during a subsequent cycle, when in the last administration of the bispecific antibody of the previous cycle, the human subject experiences CRS greater than grade 1. Any premedication may be optionally administered during the second cycle. Further premedication may be optionally administered during subsequent cycles as well.

In one embodiment, premedication and prophylaxis for CRS is administered, including an antihistamine such as diphenhydramine (e.g., at an intravenous or oral dose 50 mg, or an equivalent thereof), an antipyretic such as acetaminophen (e.g., at an oral dose of 650-1000 mg, or an equivalent thereof), and a corticosteroid such as dexamethasone (e.g., at a dose of 15 mg, or an equivalent thereof). In some embodiments, the premedication and prophylaxis is administered 30-120 minutes prior to administration of the bispecific antibody. On subsequent days 2, 3, and optionally day 4, further prophylaxis is administered comprising the systemic administration of a corticosteroid such as dexamethasone (e.g., at a dose of 15 mg, or an equivalent thereof). In some embodiments, the premedication and prophylaxis schedule preferably is administered during the first four administrations of the bispecific antibody, e.g., during the first 28-day cycle of bispecific antibody administration described herein. Furthermore, subsequent cycles, in case of, e.g., CRS greater than grade 1 occurring during the last administration of the prior cycle, can include the same administration schedule, wherein the premedication as part of the administration schedule is optional.

During the treatment of a human subject with indolent lymphoma (e.g., FL) using the doses and treatment regimens described herein, CRS can be well managed while at the same time effectively controlling and/or treating the indolent lymphoma. As described in the Examples, subjects treated with the methods described herein may experience manageable CRS. In some cases, subjects receiving the treatment described herein may develop CRS of grade 1 as defined in accordance with standard practice. In other cases, subjects may develop manageable CRS of grade 2 as defined in accordance with standard practice. Hence, subjects receiving the treatments described herein may have manageable CRS of grade 1 or grade 2 during as defined in accordance with standard practice. In accordance with standard classification for CRS, a grade 1 CRS includes a fever to at least 38° C., no hypotension, no hypoxia, and a grade 2 CRS includes a fever to at least 38° C. plus hypotension, not requiring vasopressors and/or hypoxia requiring oxygen by low flow nasal cannula or blow by. Such manageable CRS can occur during cycle 1. Human subjects receiving the treatments described herein may also have CRS greater than grade 2 during the treatments as defined in accordance with standard practice. Hence, human subjects receiving the treatments described herein may also have CRS of grade 3 during said treatments as defined in accordance with standard practice. Such manageable CRS may further occur during cycle 1 and subsequent cycles.

Human subjects treated according to the methods described herein may also experience pyrexia, fatigue, and injection site reactions. They may also experience neurotoxicity, partial seizures, agraphia related to CRS, or confusional state related to CRS.

As mentioned above, subjects may develop CRS during treatment with the methods described herein, despite having received CRS prophylaxis. CRS grading criteria are described in Tables 5 and 6.

In one embodiment, subjects who develop Grade 1 CRS are treated with antibiotics if they present with infection. In some embodiments, the antibiotics are continued until neutropenia, if present, resolves. In some embodiments, subjects with Grade 1 CRS who exhibit constitutional symptoms are treated with NSAIDs.

In one embodiments, subjects who develop Grade 2 CRS are treated with intravenous fluid boluses and/or supplemental oxygen. In some embodiments, subjects who develop Grade 2 CRS are treated with a vasopressor. In some embodiments, subjects with Grade 2 CRS with comorbidities are treated with tocilizumab (a humanized antibody against IL-6 receptor, commercially available as, e.g., ACTEMRA®) and/or steroids (e.g., dexamethasone or its equivalent of methylprednisolone). In a further embodiment, a subject who presents with concurrent ICANS is administered dexamethasone. In yet a further embodiment, if the subject does not show improvement in CRS symptoms within, e.g., 6 hours, or if the subject starts to deteriorate after initial improvement, then a second dose of tocilizumab is administered together with a dose of corticosteroids. In some embodiments, if the subject is refractory to tocilizumab after three administrations, then additional cytokine therapy, e.g., an anti-IL-6 antibody (e.g., siltuximab) or an IL-IR antagonist (e.g., anakinra) is administered to the subject.

In one embodiment, subjects who develop Grade 3 CRS are treated with vasopressor (e.g., norepinephrine) support and/or supplemental oxygen. In some embodiments, subjects with Grade 3 CRS are treated with tocilizumab, or tocilizumab in combination with steroids (e.g., dexamethasone or its equivalent of methylprednisolone). In some embodiments, a subject who presents with concurrent ICANS is administered dexamethasone. In a further embodiment, if the subject is refractory to tocilizumab after three administrations, then additional cytokine therapy, e.g., an anti-IL-6 antibody (e.g., siltuximab) or an IL-IR antagonist (e.g., anakinra) is administered to the subject.

In one embodiment, subjects who develop Grade 4 CRS are treated with vasopressor support and/or supplemental oxygen (e.g., via positive pressure ventilation, such as CPAP, BiPAP, intubation, or mechanical ventilation). In some embodiments, the subject is administered at least two vasopressors. In some embodiments, the subject is administered tocilizumab and a steroid. In a further embodiment, a subject who presents with concurrent ICANS is administered dexamethasone. In yet a further embodiment, if the subject is refractory to tocilizumab after three administrations, then additional cytokine therapy, e.g., an anti-IL-6 antibody (e.g., siltuximab) or an IL-IR antagonist (e.g., anakinra) is administered to the subject.

In some embodiments, the human subject receives prophylactic treatment for tumor lysis syndrome (TLS). Classification and grading of tumor lysis syndrome can be performed using methods known in the art, for example, as described in Howard et al. N Engl J Med 2011; 364:1844-54, and Coiffier et al., J Clin Oncol 2008; 26:2767-78. In some embodiments, prophylactic treatment of TLS comprises administering uric acid reducing agents prior to administering the bispecific antibody. Exemplary uric acid reducing agents include rasburicase and allopurinol. Accordingly, in one embodiment, the prophylactic treatment of TLS comprises administering rasburicase prior to administering the bispecific antibody. In some embodiments, when the subject shows signs of TLS, supportive therapy, such as rasburicase, may be used.

In one embodiment, the bispecific antibody is administered subcutaneously, and thus is formulated in a pharmaceutical composition such that it is compatible with subcutaneous (s.c.) administration, i.e., having a formulation and/or concentration that allows pharmaceutical acceptable s.c. administration at the doses described herein. In some embodiments, subcutaneous administration is carried out by injection. For example, formulations for DuoBody CD3×CD20 that are compatible with subcutaneous formulation and can be used in the methods described herein have been described previously (see, e.g., WO2019155008, which is incorporated herein by reference). In some embodiments, the bispecific antibody may be formulated using sodium acetate trihydrate, acetic acid, sodium hydroxide, sorbitol, polysorbate 80, and water for injection, and have a pH of 5.5 or about 5.5. In some embodiments, the bispecific antibody is provided as a 5 mg/mL or 60 mg/mL concentrate. In other embodiments, the desired dose of the bispecific antibody is reconstituted to a volume of about 1 mL for subcutaneous injection.

In one embodiment, a suitable pharmaceutical composition for the bispecific antibody can comprise the bispecific antibody, 20-40 mM acetate, 140-160 mM sorbitol, and a surfactant, such as polysorbate 80, and having a pH of 5.3-5.6. In some embodiments, the pharmaceutical formulation may comprise an antibody concentration in the range of 5-100 mg/mL, e.g., 48 or 60 mg/mL of the bispecific antibody, 30 mM acetate, 150 mM sorbitol, 0.04% w/v polysorbate 80, and have a pH of 5.5. Such a formulation may be diluted with, e.g., the formulation buffer to allow proper dosing and subcutaneous administration.

The volume of the pharmaceutical composition is appropriately selected to allow for subcutaneous administration of the antibody. For example, the volume to be administered is in the range of about 0.3 mL to about 3 mL, such as from 0.3 mL to 3 mL. The volume to be administered can be 0.5 mL, 0.8 mL, 1 mL, 1.2 mL, 1.5 ml, 1.7 mL, 2 mL, or 2.5 mL, or about 0.5 mL, about 0.8 mL, about 1 mL, about 1.2 mL, about 1.5 ml, about 1.7 mL, about 2 mL, or about 2.5 mL. Accordingly, in one embodiment, the volume to be administered is 0.5 mL or about 0.5 mL. In some embodiments, the volume to be administered is 0.8 mL or about 0.8 mL. In some embodiments, the volume to be administered is 1 mL or about 1 mL. In some embodiments, the volume to be administered is 1.2 mL or about 1.2 mL. In some embodiments, the volume to be administered is 1.5 mL or about 1.5 mL. In some embodiments, the volume to be administered is 1.7 mL or about 1.7 mL. In some embodiments, the volume to be administered is 2 mL or about 2 mL. In some embodiments, the volume to be administered is 2.5 mL or about 2.5 mL.

It is understood that the method in accordance with the invention may be the first, or may be part of the first treatment provided to such patients. However, patients may have been subjected to prior treatments of indolent lymphoma. Prior treatments may include, one or more of chemotherapy, radiation therapy, immunotherapy, and targeted therapy, or combination hereof, but not may not be restricted thereto. Most commonly the standard of care comprises treatments with CD20 monoclonal antibodies, alkylating agents, and anthracycline, either alone or in combination. It is understood that methods and uses in accordance with the invention may also be used in combination with other suitable treatments.

Hence, in a further embodiment, in a method in accordance with the invention, a human subject having indolent lymphoma has received at least one line of treatment prior to the treatment in accordance with the invention. In another embodiment, a human subject having indolent lymphoma has received one line of treatment prior to the treatment in accordance with the invention. In another further embodiment, a human subject having indolent lymphoma has received two lines of treatment prior to the treatment in accordance with the invention. In still another further embodiment, a human subject having indolent lymphoma has received three lines of treatment prior to the treatment in accordance with the invention. In yet another further embodiment, a subject having indolent lymphoma has received more than three lines of treatment prior to the treatment in accordance with the invention. In another further embodiment, a subject having indolent lymphoma has received one, two, three, or more lines of treatment prior to the treatment in accordance with the invention.

In one embodiment, the bispecific antibody used in the methods described herein comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences within the amino acid sequence of SEQ ID NO: 6, and the VL region comprises the CDR1, CDR2 and CDR3 sequences within the amino acid sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences within the amino acid sequence of SEQ ID NO: 13, and the VL region comprises the CDR1, CDR2 and CDR3 sequences within the amino acid sequence SEQ ID NO: 14.

CDR1, CDR2 and CDR3 regions can be identified from variable heavy and light chain regions using methods known in the art. The CDR regions from said variable heavy and light chain regions can be annotated according to IMGT (see Lefranc et al., Nucleic Acids Research 1999; 27:209-12, 1999] and Brochet. Nucl Acids Res 2008; 36:W503-8).

In some embodiments, the bispecific antibody comprises:
(i) a first binding arm comprising a first antigen-binding region which binds to human CD3ε (epsilon) and comprises VHCDR1, VHCDR2 and VHCDR3 the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and
(ii) a second binding arm comprising a second antigen-binding region which binds to human CD20 and comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.

In some embodiments, the bispecific antibody comprises:
(i) a first binding arm comprising a first antigen-binding region which binds to human CD3ε (epsilon) and comprises a VH region comprising the amino acid sequence of SEQ ID NO: 6, and a VL region comprising the amino acid sequence of SEQ ID NO: 7; and
(ii) a second binding arm comprising a second antigen-binding region which binds to human CD20 and comprises a VH region comprising the amino acid sequence of SEQ ID NO: 13, and a VL region comprising the amino acid sequence of SEQ ID NO: 14.

In one embodiment, the bispecific antibody is a full-length antibody and may have an inert Fc region. In some embodiments, the first binding arm for CD3 is derived from a humanized antibody, e.g., from a full-length IgG1,λ (lambda) antibody such as H1L1 described in WO2015001085, which is incorporated herein by reference, and/or the second binding arm for CD20 is derived from a human antibody, e.g., from a full-length IgG1,K (kappa) antibody such as clone 7D8 as described in WO2004035607, which is incorporated herein by reference. The bispecific antibody may be produced from two half molecule antibodies. Each of the two half molecule antibodies comprising, e.g., the respective first and second binding arms set forth in SEQ ID NOs: 24 and 25, and SEQ ID NOs: 26 and 27. The half-antibodies may be produced in CHO cells and the bispecific antibodies generated by, e.g., Fab-arm exchange. In one embodiment, the bispecific antibody is a functional variant of Duobody CD3×CD20.

Accordingly, in some embodiments, the bispecific antibody comprises (i) a first binding arm comprising a first antigen-binding region which binds to human CD3ε (epsilon) and comprises a VH region comprising an amino acid sequence which is at least 85%, 90%, 95%, 96%, 97%, 98%, or 99% identical to SEQ ID NO: 6 or a VH region comprising the amino acid sequence of SEQ ID NO: 6, but with 1, 2, or 3 mutations (e.g., amino acid substitutions), and a VL region comprising an amino acid sequence which is at least 85%, 90%, 95%, 96%, 97%, 98%, or 99% identical to SEQ ID NO: 7 or a VL region comprising the amino acid sequence of SEQ ID NO: 7, but with 1, 2, or 3 mutations (e.g., amino acid substitutions); and
(ii) a second binding arm comprising a second antigen-binding region which binds to human CD20 and comprises a VH region comprising an amino acid sequence which is at least 85%, 90%, 95%, 98%, or 99% identical to SEQ ID NO: 13 or a VH region comprising the amino acid sequence of SEQ ID NO: 13, but with 1, 2, or 3 mutations (e.g., amino acid substitutions), and a VL region comprising an amino acid sequence which is at least 85%, 90%, 95%, 98%, or 99% identical to SEQ ID NO: 14 or a VL region comprising the amino acid sequence of SEQ ID NO: 14, but with 1, 2, or 3 mutations (e.g., amino acid substitutions).

In one embodiment, the bispecific antibody comprises:
(i) a first binding arm comprising a first antigen-binding region which binds to human CD3ε (epsilon) and comprises a heavy chain comprising the amino acid sequence of SEQ ID NO: 24, and a light chain comprising the amino acid sequence of SEQ ID NO: 25; and
(ii) a second binding arm comprising a second antigen-binding region which binds to human CD20 and comprises a VH region comprising the amino acid sequence of SEQ ID NO: 26, and a VL region comprising the amino acid sequence of SEQ ID NO: 27.

In some embodiments, the bispecific antibody comprises:
(i) a first binding arm comprising a first antigen-binding region which binds to human CD3ε (epsilon) and comprises a heavy chain comprising an amino acid sequence which is at least 85%, 90%, 95%, 98%, or 99% identical to SEQ ID NO: 24 or a heavy chain comprising the amino acid sequence of SEQ ID NO: 24, but with 1, 2, or 3 mutations (e.g., amino acid substitutions), and a light chain comprising an amino acid sequence which is at least 85%, 90%, 95%, 98%, or 99% identical to SEQ ID NO: 25 or a light chain region comprising the amino acid sequence of SEQ ID NO: 25, but with 1, 2, or 3 mutations (e.g., amino acid substitutions); and
(ii) a second binding arm comprising a second antigen-binding region which binds to human CD20 and comprises a heavy chain comprising an amino acid sequence which is at least 85%, 90%, 95%, 98%, or 99% identical to SEQ ID NO: 26 or a heavy chain comprising the amino acid sequence of SEQ ID NO: 26, but with 1, 2, or 3 mutations (e.g., amino acid substitutions), and a light chain comprising an amino acid sequence which is at least 85%, 90%, 95%, 98%, or 99% identical to SEQ ID NO: 27 or a light chain region comprising the amino acid sequence of SEQ ID NO: 27, but with 1, 2, or 3 mutations (e.g., amino acid substitutions).

Various constant regions or variants thereof may be used in the bispecific antibody. In one embodiment, the antibody comprises an IgG constant region, such as a human IgG1 constant region, e.g., a human IgG1 constant region as defined in SEQ ID NO: 15, or any other suitable IgG1 allotype. In one embodiment, the first binding arm of the bispecific antibody is derived from a humanized antibody, e.g., from a full-length IgG1,λ (lambda) antibody, and thus comprises a λ light chain constant region. In some embodiments, the first binding arm comprises a λ light chain constant region as defined in SEQ ID NO: 22. In some embodiments, the second binding arm of the bispecific antibody is derived from a human antibody, preferably from a full-length IgG1,κ (kappa) antibody, and thus may comprise a κ light chain constant region. In some embodiments, the second binding arm comprises a κ light chain constant region as defined in SEQ ID NO: 23.

It is understood that the constant region portion of the bispecific antibody may comprise modifications that allow for efficient formation/production of bispecific antibodies and/or provide for an inert Fc region. Such modifications are well known in the art.

Different formats of bispecific antibodies are known in the art (reviewed by Kontermann, Drug Discov Today 2015; 20:838-47; MAbs, 2012; 4:182-97). Thus, the bispecific antibody used in the methods and uses described herein are not limited to any particular bispecific format or method of producing it. For example, bispecific antibodies may include, but are not limited to, bispecific antibodies with complementary CH3 domains to force heterodimerization, Knobs-into-Holes molecules (Genentech, WO9850431), CrossMAbs (Roche, WO2011117329), or electrostatically-matched molecules (Amgen, EP1870459 and WO2009089004; Chugai, US201000155133; Oncomed, WO2010129304).

Preferably, the bispecific antibody comprises an Fc-region comprising a first heavy chain with a first Fc sequence comprising a first CH3 region, and a second heavy chain with a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions. Further details on these interactions and how they can be achieved are provided in e.g., WO2011131746 and WO2013060867 (Genmab), which are hereby incorporated by reference. In one embodiment, the bispecific antibody comprises in the first heavy chain (i) the amino acid L in the position corresponding to F405 in the human IgG1 heavy chain constant region of SEQ ID NO: 15, and comprises in the second heavy chain the amino acid R in the position corresponding to K409 in the human IgG1 heavy chain constant region of SEQ ID NO: 15, or vice versa.

Bispecific antibodies may comprise modifications in the Fc region to render the Fc region inert, or non-activating. Thus, in the bispecific antibodies disclosed herein, one or both heavy chains may be modified so that the antibody induces Fc-mediated effector function to a lesser extent relative to the bispecific antibody which does not have the modification. Fc-mediated effector function may be measured by determining Fc-mediated CD69 expression on T cells (i.e., CD69 expression as a result of CD3 antibody-mediated, Fcγ receptor-dependent CD3 crosslinking), by binding to Fcγ receptors, by binding to C1q, or by induction of Fc-mediated cross-linking of FcγRs. In particular, the heavy chain constant region sequence may be modified so that Fc-mediated CD69 expression is reduced by at least 50%, at least 60%, at least 70%, at least 80%, at least 90%, at least 99% or 100% when compared to a wild-type (unmodified) antibody, wherein said Fc-mediated CD69 expression is determined in a PBMC-based functional assay, e.g., as described in Example 3 of WO2015001085. Modifications of the heavy and light chain constant region sequences may also result in reduced binding of C1q to said antibody. As compared to an unmodified antibody, the reduction may be by at least 70%, at least 80%, at least 90%, at least 95%, at least 97%, or 100%, and Clq binding may be determined, e.g., by ELISA. Further, the Fc region which may be modified so that the antibody mediates reduced Fc-mediated T-cell proliferation compared to an unmodified antibody by at least 50%, at least 60%, at least 70%, at least 80%, at least 90%, at least 99% or 100%, wherein said T-cell proliferation is measured in a PBMC-based functional assay. Examples of amino acid positions that may be modified, e.g., in an IgG1 isotype antibody, include positions L234 and L235. Thus, in one embodiment, the bispecific antibody may comprise a first heavy chain and a second heavy chain, and wherein in both the first heavy chain and the second heavy chain, the amino acid residues at the positions corresponding to positions L234 and L235 in a human IgG1 heavy chain according to Eu numbering are F and E, respectively. In addition, a D265A amino acid substitution can decrease binding to all Fcγ receptors and prevent ADCC (Shields et al., JBC 2001; 276:6591-604). Therefore, the bispecific antibody may comprise a first heavy chain and a second heavy chain, wherein in both the first heavy chain and the second heavy chain, the amino acid residue at the position corresponding to position D265 in a human IgG1 heavy chain according to Eu numbering is A.

In one embodiment, in the first heavy chain and second heavy chain of the bispecific antibody, the amino acids in the positions corresponding to positions L234, L235, and D265 in a human IgG1 heavy chain, are F, E, and A, respectively. An antibody having these amino acids at these positions is an example of an antibody having an inert Fc region, or a non-activating Fc region.

With regard to the bispecific antibodies described herein, those which have the combination of three amino acid substitutions L234F, L235E and D265A and in addition the K409R or the F405L mutation, as described above, may be referred to with the suffix “FEAR” or “FEAL”, respectively.

An amino acid sequence of a wild type IgG1 heavy chain constant region may be identified herein as SEQ ID NO: 15. Consistent with the embodiments disclosed above, the bispecific antibody may comprise an IgG1 heavy chain constant region carrying the F405L substitution and may have the amino acid sequence set forth in SEQ ID NO: 17 and/or an IgG1 heavy chain constant region carrying the K409R substitution and may have the amino acid sequence set forth in SEQ ID NO: 18, and have further substitutions that render the Fc region inert or non-activating. Hence, in one embodiment, the bispecific antibody comprises a combination of IgG1 heavy chain constant regions, with the amino acid sequence of one of the IgG1 heavy chain constant regions carrying the L234F, L235E, D265A and F405L substitutions (e.g., as set forth in SEQ ID NO: 19) and the amino acid sequence of the other IgG1 heavy chain constant region carrying the L234F, L235E, D265A and K409R substitutions (e.g., as set forth in SEQ ID NO: 20).

In some embodiments, the bispecific antibody used in the methods and uses described herein comprises a first binding arm comprising a heavy chain and a light chain as defined in SEQ ID NOs: 24 and 25, respectively, and a second binding arm comprising a heavy chain and a light chain as defined in SEQ ID NOs: 26 and 27, respectively. Such an antibody is referred to herein as DuoBody CD3×CD20. Also, variants of such antibodies are contemplated use in the methods and uses as described herein. In some embodiments, the bispecific antibody is epcoritamab (CAS 2134641-34-0), or a biosimilar thereof.

### Kits

Also provided herein are kits which include a pharmaceutical composition containing a bispecific antibody which binds to CD3 and CD20 in accordance with the invention, such as DuoBody CD3×CD20 or epcoritamab, and a pharmaceutically acceptable carrier, in a therapeutically effective amount adapted for use in the methods described herein. The kits optionally also can include instructions, e.g., comprising administration schedules, to allow a practitioner (e.g., a physician, nurse, or patient) to administer the composition or compositions contained therein to a patient with DLBCL. The kit also can include a syringe or syringes.

Optionally, the kits include multiple packages of the single-dose pharmaceutical compositions each containing an effective amount of the bispecific antibody for a single administration in accordance with the methods described herein. They may also include multiple packages of single dose pharmaceutical. Instruments or devices necessary for administering the pharmaceutical composition(s) also may be included in the kits.

The present disclosure is further illustrated by the following examples, which should not be construed as further limiting. The contents of all figures and all references, Genbank sequences, journal publications, patents, and published patent applications cited throughout this application are expressly incorporated herein by reference.

### EXAMPLES

### Example 1

Step-up dosing (SUD) is a strategy used to mitigate incidence and severity of CRS events. The SUD regimen usually consists of a target full dose preceded by one or more lower doses to desensitize the patient's immune system and “prime” the patients for the target full dose.

A model-based approach was applied to identify optimal step-up dosing regimens for testing of epcoritamab in DLBCL patients and FL patients. A repeated time-to-event (RTTE) model was developed using PK and CRS event data collected across EPCORE NHL-1 (NCT03625037; monotherapy) and EPCORE NHL-3 (NCT04542824; monotherapy in Japanese patients) studies (Hutchings M, et al. Lancet. 2021; 398:1157-69; Thieblemont C, et al. J Clin Oncol. 2022; DOI:10.1200/JCO.22.01725; Izutsu K, et al. JSMO 2023. Abstract O13-3).

It was apparent based on data collected during these dose-escalation study that at least 2 steps (priming and intermediate dose) are necessary to effectively mitigate the risk of CRS. Wide priming (0.004-0.16 mg) and intermediate (0.25-1.6 mg) dose ranges were explored, and the SUD dosing regimen of 0.16/0.8/48 mg was selected for expansion based on the observed incidence and severity of CRS events.

Models considered in the development of the RTTE analysis were those capable of incorporating longitudinal exposure into a survival framework. Mechanistically, risk of CRS is linked to the production of cytokines and the tumor-killing action of the compound. To capture these dynamics, the hazard function (risk of CRS event) was modeled as the product of 2 components to allow flexibility to capture onset and tolerance dynamics of epcoritamab exposure affecting the hazard of CRS events empirically.

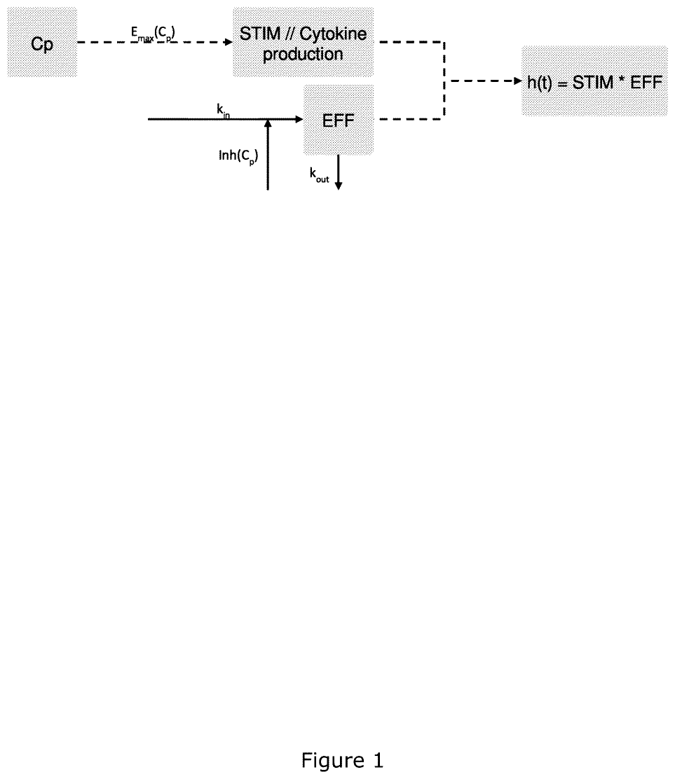

The first (STIM), which addressed the onset of AE risk, allowed for increasing hazard in some (possibly time-delayed) function of epcoritamab plasma concentration, and the second (EFF) described the inhibition of the hazard in some (again, possibly time-delayed) function of epcoritamab plasma concentration to capture tolerance dynamics. Simulations using the developed model were performed to predict the probability of grade 2+ CRS over first 2 cycles of epcoritamab treatment, and to assess different SUD regimens. In addition, a 3-step SUD regimen was assessed, and the optimal SUD regimens were identified based on the simulation results and selected for further investigation in the clinic (FIG. 1).

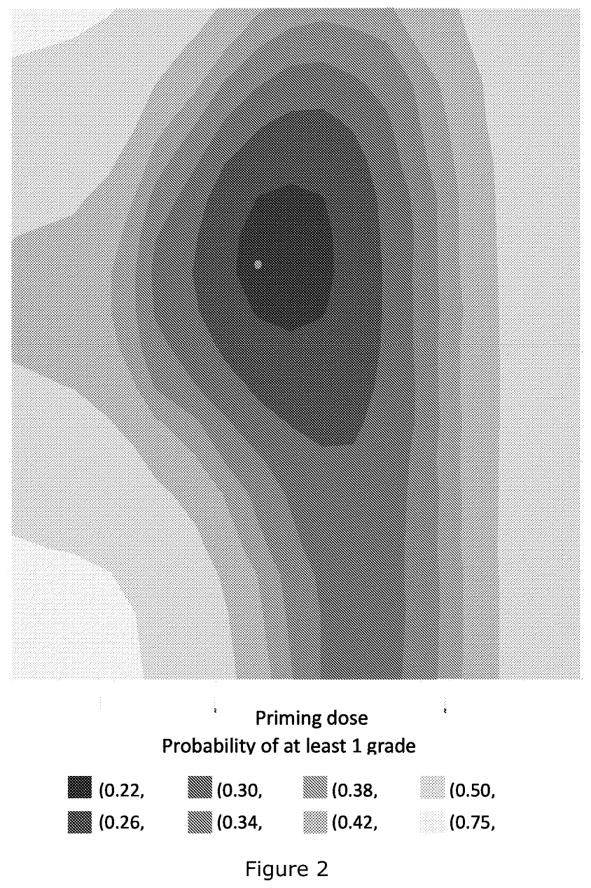

The model was used to predict risk of grade 2+ CRS at regimens beyond the priming and intermediate dose permutations tested in EPCORE NHL-1 dose escalation. Based on model prediction, further increasing the priming or intermediate dose may only lead to a small reduction in risk of grade 2+ CRS in DLBCL (FIG. 2). Based on results from the simulation, 2 alternative SUD regimens were selected to be explored in the clinic.

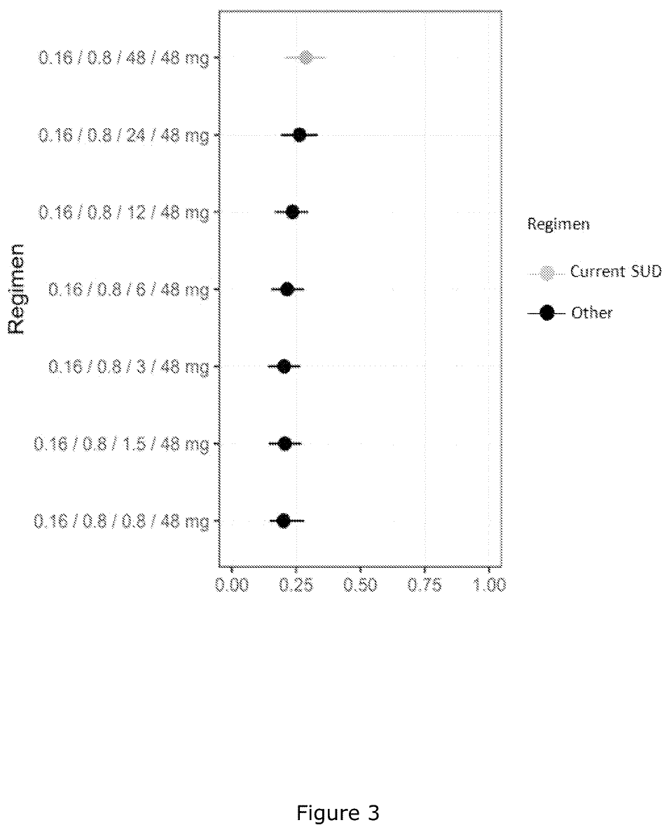

Based on model prediction, a 3-step SUD regimen can potentially lead to a reduction in risk of grade 2+ CRS (FIG. 3). It was predicted that a 3-step SUD could improve benefit-risk in less aggressive NHL (e.g., FL). Based on results from the simulation, 2 different 3-step SUD regimens were selected to be explored in FL patients.

### Example 2

### GCT3013-01: A Phase 1/2, Open-Label, Dose-Escalation Trial of GEN3013 in Patients with Relapsed, Progressive or Refractory B-Cell Lymphoma

GCT3013-01 is an open-label, multicenter, phase 1/2 trial of epcoritamab in subjects with relapsed, progressive, or refractory B-cell lymphoma. The trial includes dose expansion of epcoritamab in subjects with FL grades 1-3A (hereinafter referred to as “Pivotal Cohort”). The trial further includes an Optimization Part evaluating two additional step-up dosing (SUD) regimens of epcoritamab in subjects with FL grades 1-3A (Arm A: 0.16/0.8/3/48 mg, Arm B: 0.16/0.8/6/48 mg) and their effect on the rate of grade ≥2 cytokine release syndrome (CRS) events.

### Dosing Regimens for Epcoritamab

TABLE 2
Dosing Regimen for FL Grades 1-3A (pivotal cohort)

| Priming | Intermediate | First Full | Second Full |
|---|---|---|---|
| Dose | Dose | Dose | Dose |
| (Cycle 1 Day 1) | (Cycle 1 Day 8) | (Cycle 1 Day 15) | (Cycle 1 Day 22) |
| 0.16 mg | 0.8 mg | 48 mg | 48 mg |

TABLE 3
Optimization Regimens for FL Grades 1-3A

| | | First | Second | First | Second |
|---|---|---|---|---|---|
| | Priming | Intermediate | Intermediate | Full | Full |
| | Dose | Dose | Dose | Dose | Dose |
| | (Cycle 1 | (Cycle 1 | (Cycle 1 | (Cycle 1 | (Cycle 2 |
| | Day 1) | Day 8) | Day 15) | Day 22) | Day 1) |
| Arm A | 0.16 mg | 0.8 mg | 3 mg | 48 mg | 48 mg |
| Arm B | 0.16 mg | 0.8 mg | 6 mg | 48 mg | 48 mg |

Epcoritamab is administered in:
Cycle 1-3: Days 1, 8, 15, and 22 (once weekly)
Cycle 4-9: Days 1 and 15 (every 2 weeks)
Cycles 10 to PD, unacceptable toxicity, or end of trial: Day 1 (every 4 weeks)

Decision rules based on the rate of grade ≥2 CRS, safety, PK and PD data are used to identify a single, preferred SUD regimen. Additional amendments to the protocol included recommendations for proper hydration and use of dexamethasone (15 mg), instead of prednisolone or other corticosteroid equivalent, as the prophylactic corticosteroid administered to minimize CRS during treatment with epcoritamab in Cycle 1.

For FL grades 1-3A optimization subjects are enrolled in 2 arms in parallel, with up to approximately 10 subjects in each arm. The dose optimization for FL grades 1-3A will assess 2 alternative second intermediate doses in parallel. The decision rule based on CRS after Stage 1 is as follows:
If ≤2 of 10 subjects experience CRS events of Grade ≥2, the SUD regimen will be considered acceptable for further evaluation.
If ≥3 of 10 subjects experience CRS events of Grade ≥2, the SUD escalation will be terminated.

### CRS Prophylaxis

Administration of corticosteroids for four days is performed to reduce/prevent the severity of symptoms from potential CRS for each dose of epcoritamab. For administration of epcoritamab in cycle 2 and beyond, CRS prophylaxis with corticosteroids is optional. Corticosteroid administration can be either intravenous or oral route with recommended dose or equivalent.

TABLE 4
Corticosteroid Dose Equivalents—Conversion Table

| | Approximate equivalent |
|---|---|
| Glucocorticoid | dose (mg) |
| Short-acting | |
| Cortisone (PO) | 500 |
| Hydrocortisone (IV or PO) | 400 |
| Intermediate-acting | |
| Methylprednisolone (IV or PO) | 80 |
| Prednisolone (PO) | 100 |
| Prednisone (IV or PO) | 100 |
| Triamcinolone (IV) | 80 |
| Long-acting | |
| Betamethasone (IV) | 15 |
| Dexamethasone (IV or PO) | 15 |

### Supportive Care for Cytokine Release Syndrome

CRS is graded according to the ASTCT grading for CRS (Tables 6 and 7), and for treatment of CRS, subjects should receive supportive care. Supportive care can include, but is not limited to,
Infusion of saline
Systemic glucocorticosteroid, antihistamine, antipyrexia
Support for blood pressure (vasopressin, vasopressors)
Support for low-flow and high-flow oxygen and positive pressure ventilation
Monoclonal antibody against IL-6R, e.g., IV administration of tocilizumab
Monoclonal antibody against IL-6, e.g., IV siltuximab if not responding to repeated tocilizumab.

TABLE 5
Grading and Management of Cytokine Release Syndrome
Harmonized definitions and grading criteria for CRS,
per the American Society for Transplantation and Cellular
Therapy (ASTCT), formerly American Society for Blood and
Marrow Transplantation, (ASBMT), are presented below.
Grading of Cytokine Release Syndrome

| CRS | | | | | |
|---|---|---|---|---|---|
| parameter | Grade 1 | Grade 2 | Grade 3 | Grade 4 | Grade 5 |
| Fever¹ | ≥38.0° C. | ≥38.0° C. | ≥38.0° C. | ≥38.0° C. | Death due |
| With | None | Not requiring | Requiring | Requiring | to CRS in |
| hypotension | | vasopressors | 1 vasopressor | ≥2 vasopressors | which another |
| | | | with or | (excluding | cause is not |
| | | | without | vasopressin) | the principle |
| | | | vasopressin | | factor |
| And/or | None | Requiring | Requiring | Requiring | leading to |
| hypoxia² | | low-flow | high-flow | positive pressure | this outcome |
| | | (≤6 L/minute) | (>6 L/minute) | ventilation³ | |
| | | nasal cannula | nasal cannula, | (eg, CPAP, | |
| | | or blow-by | facemask, | BIPAP, | |
| | | | nonrebreather | intubation and | |
| | | | mask, or | mechanical | |
| | | | venturi mask | ventilation) | |

Abbreviations:
BiPAP, Bilevel positive airway pressure;
CPAP, continuous positive airway pressure;
CRS, cytokine release syndrome;
IV, intravenous.
Note:
organ toxicities or constitutional symptoms associated with CRS may be graded according to CTCAE but they do not influence CRS grading.
¹Fever is defined as temperature ≥38.0° C. not attributable to any other cause, with or without constitutional symptoms (eg, myalgia, arthralgia, malaise). In subjects who have CRS receiving antipyretics, anticytokine therapy, and/or corticosteroids, fever is no longer required to grade subsequent CRS severity. In this case, CRS grading is driven by hypotension and/or hypoxia.
²CRS grade is determined by the more severe event: hypotension or hypoxia not attributable to any other cause. For example, a subject with temperature of 39.5° C., hypotension requiring 1 vasopressor, and hypoxia requiring low-flow nasal cannula is classified as grade 3 CRS. Both systolic blood pressure and mean arterial pressure are acceptable for blood pressure measurement. No specific limits are required, but hypotension should be determined on a case-by-case basis, accounting for age and the subject's individual baseline, i.e., a blood pressure that is below the normal expected for an individual in a given environment.
³Intubation of a subject without hypoxia for the possible neurologic compromise of a patent airway alone or for a procedure is not by definition grade 4 CRS.
Source: Adapted from Lee et al., Biol Blood Marrow Transplant 2019; 25: 625-638

TABLE 6
Grading and Management of Cytokine Release Syndrome

| CRS | |
|---|---|
| grade | Management |
| 1 | Fever: Patients with a new fever should be admitted |
| | to the hospital if not already. Investigate for infection |
| | and rapidly startup broad-spectrum antibiotics. |
| | Continuation of antibiotic therapy is recommended |
| | until and potential neutropenia resolve. |
| | Constitutional symptoms may be helped by NSAIDs. |
| | Tocilizumab: No*. |
| | Steroids: No. |
| 2 | Fever: As per grade 1. |
| | Hypotension: Immediate clinical evaluation and |
| | intervention is warranted. At the first confirmed |
| | decrease ≥20% from baseline systolic, diastolic or mean |
| | arterial pressure or evidence of worsening perfusion, |
| | administer an IV fluid bolus (20 ml/kg up to 1 L). |
| | Consider a vasopressor and administer no |
| | later than after the 3^{rd} IV fluid bolus due the |
| | vasodilatation and capillary leak associated with CRS. |
| | Hypoxia: Consider X-ray or CT-scan if hypoxic and/ |
| | or tachypneic. Administer oxygen by |
| | low-flow nasal cannula (≤6 L/min) or blow-by. |
| | Tocilizumab: No* (yes, if the patient has comorbidities^{†}). |
| | Steroids: No (consider, if the patient has comorbidities^{‡}). |
| 3 | Fever: As per grade 1. |
| | Hypotension: Immediate clinical evaluation and |
| | intervention is warranted. Administer a vasopressor |
| | (norepinephrine), with or without vasopressin, as most |
| | patients with CRS have peripheral vasodilation. |
| | Hypoxia: Administer oxygen by high-flow nasal |
| | cannula (>6 L/min), facemask, non- |
| | breather mask, or Venturi mask. |
| | Tocilizumab: Yes^{†}. |
| | Steroids: Consider^{‡}. |
| 4 | Fever: As per grade 1. |
| | Hypotension: Immediate clinical evaluation and |
| | intervention is warranted. Administer at least 2 |
| | vasopressors, with or without vasopressin, as most |
| | patients with CRS have peripheral vasodilation. |
| | Hypoxia: Positive pressure (e.g., CPAP, BiPAP, |
| | intubation, and mechanical ventilation). |
| | Tocilizumab: Yes^{†}. |
| | Steroids: Yes^{‡}. |

*Consider intervening earlier in specific cases. For example, an elderly patient with prolonged fever (>72 hours) or very high fever (>40.5° C./104.9° F.) may not tolerate the resulting sinus tachycardia as well as a younger patient, so tocilizumab may be indicated.
^{†}Tocilizumab (anti-IL-6R) remains the only first-line anticytokine therapy approved for CRS. If there is no improvement in symptoms within 6 hours, or if the patient starts to deteriorate after initial improvement, a second dose of tocilizumab should be administered along with a dose of corticosteroids. For patients being refractory to tocilizumab (3 administrations), additional anticytokine therapy such as siltuximab (anti-IL-6) or anakinra (anti-IL-1R) may be considered. However, such use is entirely anecdotal and, as such, is entirely at the discretion of the treating physician.
^{‡}Consider dexamethasone over methylprednisolone due to improved CNS penetration even in absence of neurotoxicity, as high-grade CRS is correlated with risk of concurrent or subsequent ICANS. If concurrent ICANS is observed, dexamethasone should be preferred.
Source: Varadarajan I, Kindwall-Keller TL, Lee DW (2020). Management of cytokine release syndrome. In: Chimeric antigen receptor T-cell therapies for cancer (Chapter 5). Elsevier 2020

### Tumor Lysis Syndrome Prevention and Management

For prophylactic treatment of tumor lysis syndrome, subjects receive hydration and uric acid reducing agents prior to the administration of epcoritamab. If signs of tumor lysis syndrome (TLS) occur, supportive therapy, including rasburicase, is used.

### Study Assessments

### Demographics and Baseline Assessments

Demographic details of subjects are collected, as is information such as date of lymphoma diagnosis, Ann Arbor Staging at diagnosis, including constitutional symptoms (B symptoms), and prior evidence of CD20 positivity. Medical history, information regarding prior and concomitant medications, concomitant procedures, and prior cancer therapies and surgeries (including prior anti-cancer therapy for NHL, such as surgery, radiotherapy, chemo-radiotherapy, and systemic treatment regimens), are also collected.

### Efficacy Assessments

Eligible subjects have at least 1 measurable site of disease (as indicated in the inclusion criteria) for disease evaluations. Measurable sites of lymphoma are defined as lymph nodes, lymph node masses, or extranodal sites. Measurements are determined by imaging evaluation, with up to 6 measurable sites followed as target lesions for each subject. Sites not measurable as defined above are considered assessable by objective evidence of disease (i.e., radiographic imaging, physical examination, or other procedures). Examples of assessable disease include, e.g., bone marrow involvement, bone lesions, effusions, or thickening of bowel wall.

### Tumor and Bone Marrow Biopsies

Two fresh core tumor biopsies are collected before treatment with epcoritamab (during the screening period) and 2 fresh core tumor biopsies at the start of cycle 2 day 15 (±1 week) for all subjects with accessible tumors. An archival tumor biopsy, if collected within 3 months prior to enrollment, is acceptable if a fresh biopsy at screening cannot be collected. The biopsy can be a whole lymph node or a core biopsy. Tumor biopsies should be FFPE. Tumor biopsies are examined for MRD assessment and exploratory biomarkers.

### Radiographic Assessments

An FDG PET-CT scan (or CT/MRI and FDG PET when PET-CT scan not available) is performed during Screening. For subjects with FDGavid tumors at Screening, all subsequent disease assessments include FDGPET using the 5-point scale described in Barrington et al. (J Clin Oncol 2014; 32:3048-58; Score 1: No uptake; Score 2: Uptake ≤mediastinum; Score 3: Update >mediastinum but ≤liver; Score 4: Uptake moderately higher than liver; Score 5: Uptake markedly higher than liver and/or new lesions; Score X: new areas of update unlikely to be related to lymphoma). For subjects with non-avid or variably FDG-avid tumors, CT scan with IV contrast of neck/chest/abdomen/pelvis/additional known lesions may be performed. The CT component of the PET-CT may be used in lieu of a standalone CT/MRI, if the CT component is of similar diagnostic quality as a contrast enhanced CT performed without PET. If contrast enhanced PETCT is not available, a standalone diagnostic CT/MRI and a standard FDGPET is performed. Subjects who are intolerant of IV CT contrast agents undergo CT scans with oral contrast.

MRI can be used to evaluate sites of disease that cannot be adequately imaged using CT or for subjects intolerant of CT contrast agents. In cases where MRI is the imaging modality of choice, the MRI is obtained at screening and at all subsequent response evaluations.

### Bone Marrow Assessments

A bone marrow biopsy (archival or fresh), with or without aspirate, is obtained at screening for all patients to document bone marrow involvement with lymphoma. A bone marrow biopsy obtained as routine SOC may be used if taken up to 42 days before first dose of epcoritamab. If bone marrow aspirate is obtained, determination of bone marrow involvement can be confirmed by flow cytometry. A bone marrow biopsy is taken (1) at screening; (2) for subjects with bone marrow involvement at screening who later achieve CR by imaging—bone marrow evaluation includes morphological examination and either flow cytometry or IHC, if warranted, to confirm the presence or absence (complete remission) of lymphoma; (3) for subjects with bone marrow involvement documented at screening who later achieve CR by imaging—a portion of the aspirate collected to confirm CR will be used for MRD assessments.

### Minimal Residual Disease Assessment

MRD is assessed by tracking the presence of DNA that encodes the B cell receptor (BCR) expressed specifically by the cancer cells. The DNA sequence of this BCR is identified by tumor biopsy submitted at screening. After the start of treatment, blood samples are taken at fixed timepoints and at the time of CR to assess whether the amount of cancer DNA is declining, as a potential measure of (early) response, and to assess MRD. As an exploratory analysis, when a subject reaches a metabolic/radiologic CR and has bone marrow involvement documented at screening, a portion of the aspirate collected to confirm CR is used to assess MRD.

### Disease Response and Progressive Disease Assessment

Disease response is assessed according to both Lugano criteria (described in Cheson et al., J Clin Oncol 2014; 32:3059-68 (see, in particular, Table 3 in Cheson et al., 2014) and LYRIC (Table 7) to inform decisions on continuation of treatment.

Endpoint definitions are as follows:

Overall response rate (ORR), is defined as the proportion of subjects who achieve a response of PR or CR, prior to initiation of subsequent therapy.

Time to response (TTR), is defined among responders, as the time between first dose (from day 1, cycle 1) of epcoritamab and the initial documentation of PR or CR.

Duration of response (DOR), is defined among responders, as the time from the initial documentation of PR or CR to the date of disease progression or death, whichever occurs earlier.

Progression-free survival (PFS), is defined as the time from the first dosing date (day 1, cycle 1) of epcoritamab and the date of disease progression or death, whichever occurs earlier.

Overall survival (OS), is defined as the time from the first dosing date (day 1, cycle 1) of epcoritamab and the date of death.

Time to next anti-lymphoma therapy (TTNT), is defined as the number of days from day 1 of cycle 1 to the first documented administration of subsequent anti-lymphoma therapy.

MRD negativity rate, is defined as the proportion of subjects with at least 1 undetectable MRD result according to the specific threshold, prior to initiation of subsequent therapy.

Lugano criteria (see, e.g., Cheson et al., J Clin Oncol 2014; 32:3059-68, for definitions of complete response, partial response, no response/stable disease, and progressive disease).

### (a) Target and Non-Target Lesions

Target lesions for the Lugano criteria include up to 6 of the largest dominant nodes, nodal masses, or other lymphomatous lesions that are measurable in two diameters and are preferably from different body regions representative of the subject's overall disease burden, including mediastinal and retroperitoneal disease, where applicable. At baseline, a measurable node is >15 mm in longest diameter (LDi). Measurable extranodal disease may be included in the six representative target lesions. At baseline, measurable extranodal lesions should be >10 mm in LDi.

All other lesions (including nodal, extranodal, and assessable disease) may be followed as non-target lesions (e.g., cutaneous, GI, bone, spleen, liver, kidneys, pleural or pericardial effusions, ascites, bone, bone marrow).

### (b) Split Lesions and Confluent Lesions

Lesions may split or may become confluent over time. In the case of split lesions, the individual product of the perpendicular diameters (PPDs) of the nodes should be summed together to represent the PPD of the split lesion; this PPD is added to the sum of the PPDs of the remaining lesions to measure response. If subsequent growth of any or all of these discrete nodes occurs, the nadir of each individual node is used to determine progression. In the case of confluent lesions, the PPD of the confluent mass should be compared with the sum of the PPDs of the individual nodes, with more than 50% increase in PPD of the confluent mass compared with the sum of individual nodes necessary to indicate progressive disease (PD). The LDi and smallest diameter (SDi) are no longer needed to determine progression.

### LYRIC

Clinical studies have shown that cancer immunotherapies may result in early apparent radiographic progression (including the appearance of new lesions), followed by a delayed response. As this initial increase in tumor size might be caused by immune-cell infiltration in the setting of a T-cell response, this progression may not be indicative of true disease progression and is therefore called “pseudoprogression” (Wolchok et al., Clin Cancer Res 2009; 15:7412-20).

The current Lugano response assessment criteria (Cheson et al., J Clin Oncol 2014; 32:3059-68) does not take pseudoprogression into account, and there is a significant risk of premature discontinuation of a potentially efficacious immunomodulatory drug following the observation of an atypical response. Atypical responses are characterized either by the early progression of existing lesions, later followed by response, or by the development of new lesions, with or without tumor shrinkage elsewhere.

LYRIC is a modification of the Lugano response assessment criteria, which has been adapted to immune-based therapies, and it implements a new, mitigating response category: the “indeterminate response” (IR) designation (Cheson et al., Blood 2016; 128:2489-96). This IR designation was introduced to potentially identify “atypical response” cases until confirmed as flare/pseudoprogression or true PD by either biopsy or subsequent imaging.

A subject who shows PD according Lugano criteria/classification will be considered to have IR in 1 or more of the 3 following circumstances:

IR (1): Increase in overall tumor burden (as assessed by sum of the product of the diameters [SPD]) of ≥50% of up to 6 target lesions in the first 12 weeks of therapy, without clinical deterioration.

IR (2): Appearance of new lesions or growth of one or more existing lesion(s) ≥50% at any time during treatment; occurring in the context of lack of overall progression (SPD <50% increase) of overall tumor burden, as measured by SPD of up to 6 lesions at any time during the treatment.

IR (3): Increase in FDG uptake of 1 or more lesion(s) without a concomitant increase in lesion size or number.

It is possible that, at a single time point, a subject could fulfill criteria for both IR (1) or IR (2) and IR (3): for example, there could be a new FDG-avid lesion in the absence of overall progression (IR [2]), and, at the same time, increase in FDG uptake of a separate lesion (IR [3]). In such cases, the designation of IR (1) or IR (2) should take priority (e.g., IR [2] in the above example).

Subjects categorized as having any of the IR types receive repeat imaging after an additional 12 weeks (or earlier if clinically indicated). At that time, response should be re-evaluated, and the subject should be considered to have true PD with the following considerations:

Follow-up IR (1); In case of IR (1), comparison should be made between the first IR (1) and the current SPD. The IR (1) will become PD if: (a) SPD increases by ≥10% from first IR1 AND (b) an increase of ≥5 mm (in either dimension) of ≥1 lesion for lesions ≤2 cm and ≥10 mm for lesions >2 cm, to be consistent with Lugano criteria.

Follow-up IR (2); In case of IR (2), the new or growing lesion(s) is added to the target lesion(s), up to a total of no more than 6 total lesions. The IR (2) will become PD if: (a) ≥50% increase in SPD (newly defined set of target lesions) from nadir value.

Follow-up IR (3); The IR (3) will become PD if lesion with increased FDG uptake also shows size increase.

TABLE 7
LYRIC

| | CR | PR | SD | PD |
|---|---|---|---|---|
| LYRIC | Same as Lugano | Same as Lugano | Same as Lugano | As with Lugano with the following |
| | Classification | Classification | Classification | exceptions: |
| | | | | IR Categories: |
| | | | | IR (1): ≥50% increase in SPD in |
| | | | | first 12 weeks of therapy |
| | | | | IR (2): <50% increase in SPD with |
| | | | | a) New lesion(s), or |
| | | | | b) ≥50% increase of 1 lesion or |
| | | | | set of lesions at any time |
| | | | | during treatment |
| | | | | IR (3): Increase in FDG uptake |
| | | | | without a concomitant increase in |
| | | | | lesion size meeting criteria for PD |

### Clinical Safety Assessments

Safety is assessed by measuring adverse events, laboratory test results, ECGs, vital sign measurements, physical examination findings, and ECOG performance status. Also assessed are immune effector cell-associated neurotoxicity syndrome (e.g., as described by Lee et al., Biol Blood Marrow Transplant 2019; 25:625-638), constitutional symptoms (B symptoms), tumor flare reaction, and survival.

### Summary of Safety Data

By April 2023, 16 subjects with R/R FL in the optimization arms had received at least 1 dose of epcoritamab (i.e., priming) and 11 subjects with FL had received at least one full dose of epcoritamab. Of the 11 subjects who had received the first full dose of epcoritamab, 6 subjects were enrolled in Arm A and 5 subjects were enrolled in Arm B. Only Grade 1 CRS events were recorded. Four (36.4%) subjects had Grade 1 CRS events reported (1 in Arm A and 3 in Arm B). One subject in Arm B had a Grade 1 CRS event occurring after the second intermediate (6 mg) dose and 2 subjects had a Grade 1 CRS event after the first full dose (1 in Arm A and 1 in Arm B).

These data indicated that adding a second intermediate dose, in conjunction with the prophylactic measures taken, mitigate the CRS risk of epcoritamab.

After 6 subjects were enrolled in each arm, preliminary analysis revealed a numerically lower CRS rate in Arm A as compared to Arm B; further enrollment in Arm A was then prioritized.

As of a data cut-off of 8 Jan. 2024, 86 subjects in Arm A and 6 subjects in Arm B received at least 1 dose of epcoritamab. Overall, 64 (74.4%) subjects in Arm A continued to receive epcoritamab treatment as of the DCO date and 22 (25.6%) subjects discontinued treatment (17 subjects discontinued treatment due to progressive disease and 3 subjects discontinued treatment due to an AE). An overview of TEAEs for all subjects is presented in Table 8.

TABLE 8
Overview of TEAEs in Subjects with R/R FL
Administered a 3-Step SUD Regimen—FL Cohort,
Optimization Part, GCT3013-01 Trial (Safety Analysis Set)

| | Arm A^{a} | Arm B^{b} |
|---|---|---|
| Number of subjects, n (%) | (N = 86) | (N = 6) |
| Number of subjects with ≥1 | | |
| TEAE | 85 (98.8%) | 6 (100%) |
| Related TEAE | 78 (90.7%) | 5 (83.3%) |
| Grade 3 and higher TEAE | 46 (53.5%) | 3 (50.0%) |
| Grade 3 and higher related TEAE | 29 (33.7%) | 2 (33.3%) |
| TEAE by worst toxicity grade | | |
| 1 | 11 (12.8%) | 0 |
| 2 | 28 (32.6%) | 3 (50.0%) |
| 3 | 32 (37.2%) | 2 (33.3%) |
| 4 | 14 (16.3%) | 1 (16.7%) |
| 5 | 0 | 0 |
| Serious TEAE | 38 (44.2%) | 3 (50.0%) |
| Serious related TEAE | 31 (36.0%) | 3 (50.0%) |
| TEAE leading to treatment discontinuation | 3 (3.5%) | 0 |
| TEAE leading to dose delay | 50 (58.1%) | 4 (66.7%) |
| Fatal TEAE | 0 | 0 |
| Fatal related TEAE | 0 | 0 |
| AESI | | |
| CRS | 42 (48.8%) | 3 (50.0%) |
| ICANS | 0 | 0 |
| CTLS | 0 | 0 |

AESI = adverse event of special interest;
CRS = cytokine release syndrome;
CTLS = clinical tumor lysis syndrome;
FL = follicular lymphoma;
ICANS = immune effector cell-associated neurotoxicity syndrome;
TEAE = treatment-emergent adverse event.
Note:
Percentages calculated based on number of subjects in Safety Analysis Set. Adverse events are classified using Medical Dictionary for Regulatory Activities v26.1 and National Cancer Institute-Common Terminology Criteria for Adverse Events v5.0 and are counted only once per category. ICANS and CRS are graded according to Lee, D. W., Santomasso, B. D., Locke, F. L., Ghobadi, A., Turtle, C. J., Brudno, J. N., Maus, M. V., Park, J. H., Mead, E., Pavletic, S., et al. (2019) .. Biol Blood Marrow Transplant 25, 625-638 and CTLS according to Cairo-Bishop (Coiffier, B., Altman, A., Pui, C. H., Younes, A., and Cairo, M. S. (2008). J Clin Oncol 26, 2767-2778).
^{a}Arm A: Epcoritamab 3-step SUD regimen (0.16/0.8/3/48 mg).
^{b}Arm B: Epcoritamab 3-step SUD regimen (0.16/0.8/6/48 mg).

Eighty-five (98.8%) subjects in Arm A experienced at least 1 TEAE, and 78 (90.7%) subjects experienced at least 1 TEAE considered related to epcoritamab by the investigator. The most common (≥20%) TEAEs by PT were CRS (48.8%), injection site reaction (26.7%), and constipation (20.9%).

A total of 46 (53.5%) subjects in Arm A experienced at least 1 grade 3 or higher TEAE; of these, 29 subjects experienced at least 1 grade 3 or higher TEAE considered related to epcoritamab by the investigator (Table 9). In Arm A, grade 3 or 4 TEAE PTs that occurred in more than 1 subject each were neutropenia (14 subjects; 16.3%), lymphocyte count decreased (6 subjects; 7.0%), lymphopenia and COVID-19 (4 subjects each; 4.7%), neutrophil count decreased (3 subjects; 3.5%), and anemia, thrombocytopenia, ALT increased, hyperglycemia, and hypokalemia (2 subjects each; 2.3%). The most common (≥2 subjects) grade 3 or 4 TEAEs considered related to epcoritamab were: neutropenia (11 subjects; 12.8%), lymphocyte count decreased (4 subjects; 4.7%), lymphopenia (3 subjects; 3.5%), neutrophil count decreased (3 subjects; 3.5%), and ALT increased (2 subjects; 2.3%).

Serious TEAEs were reported in 38 (44.2%) subjects in Arm A and were considered related to epcoritamab by the investigator in 31 subjects. The most common (≥20%) serious TEAE was CRS (24 subjects; 27.9%). Three (3.5%) subjects in Arm A had at least 1 TEAE that led to discontinuation of epcoritamab. One (1.2%) subject experienced grade 2 bronchopulmonary aspergillosis, and 2 (2.3%) subjects had grade 2 pneumonitis. No subject had a fatal (grade 5) TEAE. One subject in Arm A died due to disease progression 19 days after the last dose of epcoritamab.

AESIs included CRS, ICANS, and CTLS. Forty-two (48.8%) subjects in Arm A experienced at least 1 event of CRS (Table 9). Thirty-four (39.5%) subjects had CRS that was a maximum of grade 1 in severity, 8 (9.3%) subjects had CRS that was a maximum of grade 2 in severity, and there were no grade 3 or higher CRS events.

Overall, 42 subjects in Arm A had a total of 69 CRS events (Table 9). The median time to CRS onset from most recent epcoritamab dose across all doses for these 69 CRS events was 4.0 days (range: 1, 8). All 69 CRS events resolved with a median time to resolution of 2.0 days (range: 1, 14). No subject had ICANS or CTLS (Table 8).

TABLE 9
CRS in Subjects with R/R FL Administered a 3-Step
SUD Regimen—FL Cohort,
Optimization Part, GCT3013-01 Trial (Safety Analysis Set)

| | Arm A | Arm B |
|---|---|---|
| Number of subjects (%) | (N = 86) | (N = 6) |
| Subjects with at least 1 CRS event | 42 (48.8%) | 3 (50.0%) |
| Grade 1 | 34 (39.5%) | 3 (50.0%) |
| Grade 2 | 8 (9.3%) | 0 |
| Grade 3 | 0 | 0 |
| Grade 4 | 0 | 0 |
| Grade 5 | 0 | 0 |
| All grade | 42 (48.8%) | 3 (50.0%) |
| (95% CI) | (37.9%, 59.9%) | (11.8%, 88.2%) |
| ≥grade 2 | 8 (9.3%) | 0 |
| (95% CI) | (4.1%, 17.5%) | (0.0%, 45.9%) |
| Occurrence of any CRS signs | 42 (100%) | 3 (100%) |
| and symptoms^{a} | | |
| Fever | 42 (100%) | 3 (100%) |
| Hypotension | 6 (14.3%) | 0 |
| Hypoxia | 2 (4.8%) | 0 |
| Other | 10 (23.8%) | 1 (33.3%) |
| Subject requiring oxygen^{a} | 2 (4.8%) | 0 |
| Subject requiring vasopressor | 0 | 0 |
| (excluding midodrine/ | | |
| midodrine hydrochloride, | | |
| milrinone, vasopressin)^{a} | | |
| Subject requiring vasopressin^{a} | 0 | 0 |
| Subject with CRS^{a} | | |
| Treated with anti-cytokine therapy | 10 (23.8%) | 0 |
| Tocilizumab | 10 (23.8%) | 0 |
| Other anti-cytokine | 0 | 0 |
| Treated with corticosteroid for CRS | 11 (26.2%) | 1 (33.3%) |
| Leading to dose delay | 16 (38.1%) | 0 |
| Leading to treatment discontinuation | 0 | 0 |
| Time to CRS onset from | | |
| most recent dosing (hours) | | |
| Number of subjects | 42 | 3 |
| Median | 40.1 | 32.4 |
| Min, max | 6, 163 | 23, 33 |
| Time to CRS resolution (hours)^{a} | | |
| Subjects with resolved CRS^{a} | 42 (100%) | 3 (100%) |
| Median^{b} | 48.0 | 18.0 |
| Min, max | 1, 322 | 12, 120 |
| Time to CRS onset from | | |
| most recent dosing (days) | | |
| Number of events | 69 | 5 |
| Median | 4.0 | 2.0 |
| Min, max | 1, 8 | 2, 5 |
| Time to CRS resolution (days) | | |
| Number of resolved CRS events | 69 (100%) | 5 (100%) |
| Median | 2.0 | 2.0 |
| Min, max | 1, 14 | 1, 5 |

AESI = adverse event of special interest;
CI = confidence interval;
CRS = cytokine release syndrome;
FL = follicular lymphoma;
max = maximum;
min = minimum;
R/R = relapsed/refractory;
SUD = step-up dosing.
Note:
CRS events are graded according to Lee, D. W., Santomasso, B. D., Locke, F. L., Ghobadi, A., Turtle, C. J., Brudno, J. N., Maus, M. V., Park, J. H., Mead, E., Pavletic, S., et al. (2019). Biol Blood Marrow Transplant 25, 625-638. The toxicity grade refers to the worst toxicity grade. For a subject with multiple events, a subject is defined as resolved if all events are resolved.
Arm A: Epcoritamab 3-step SUD regimen (0.16/0.8/3/48 mg).
Arm B: Epcoritamab 3-step SUD regimen (0.16/0.8/6/48 mg).
^{a}Percentage calculated based on subjects with at least 1 CRS event.
^{b}Based on longest recorded CRS duration in subjects with >1 CRS event.

TABLE 10
Subject-Level Summary of CRS in Subjects in with R/R FL,
Pivotal Cohort and Optimization Part, Arms A and B

| | Pivotal | Optimization | Optimization |
|---|---|---|---|
| | Cohort | Part Arm A | Part Arm B |
| Number of Subjects (%) | (N = 128) | (N = 86) | (N = 6) |
| Subjects with ≥1 CSR event | 85 (66.4%) | 42 (48.8%) | 3 (50%) |
| Grade 1 | 51 (39.8%) | 34 (39.5%) | 3 (50%) |
| Grade 2 | 32 (25.0%) | 8 (9.3%) | 0 |
| Grade 3 | 2 (1.6%) | 0 | 0 |
| Grade 4 | 0 | 0 | 0 |
| Grade 5 | 0 | 0 | 0 |

### Summary of Efficacy Data

### Pivotal Cohort

More than half of the subjects with FL (N=128) were male (79 subjects; 61.7%). The median age was 65.0 years (range: 39, 84), with 67 (52.3%) subjects ≥65 years old and 17 (13.3%) subjects ≥75 years old. More than half of subjects (77 subjects; 60.2%) were White. Most subjects with FL had advanced stage lymphoma (Ann Arbor stages III-IV in 85.2% of subjects), a FLIPI score ≥3 (60.9% of subjects) and were double refractory to both an anti-CD20 and alkylating agent (70.3% of subjects). In addition, subjects were heavily pretreated, having received a median of 3.0 (range: 2, 9) prior lines of systemic anti lymphoma therapy and 52.3% of subjects with FL had experienced progression of disease within 24 months (POD24) from any first line therapy. Thus, the FL subjects treated in the trial represent a population that is clinically very challenging to treat, with historically poor responses and survival outcomes.

The ORR was 82.0% (95% CI: 74.3, 88.3) and the CR rate was 62.5% (95% CI: 53.5, 70.9) in subjects with FL. Efficacy was consistent across prespecified subgroups, including elderly and heavily pre-treated subjects, as well as those with double refractory or POD24 disease.

After a median DOR follow-up of 14.8 months (range: 0.0+, 27.2+), the median DOR and duration of complete response (DOCR) for subjects with FL were not reached. The estimated percentage of subjects remaining in response at 12 months was 68.7% (all responders) and 82.2% (complete responders).

Median time to response (TTR) was 1.4 months (range: 1.0, 3.0) for all responders with FL and median time to complete response (TTCR) was 1.5 months (range: 1.2, 11.1) for complete responders. Median PFS (primary definition) was 15.4 months (95% CI: 10.9, NR) in subjects with FL. Median PFS was not reached (95% CI: 22.8, NR) for subjects with CR.

With a median follow-up of 17.4 months, the median OS in subjects with FL was not reached, with an estimated 81.1% of subjects alive at 12 months.

### Optimization Part

A total of 92 subjects received at least one dose of epcoritamab in the FL Optimization Part, including 86 subjects assigned to receive the proposed dosing regimen of epcoritamab 0.16/0.8/3/48 mg (Arm A) and 6 subjects assigned to receive epcoritamab 0.16/0.8/6/48 mg (Arm B). In the following, data from both Arm A and Arm B are presented, but focus is primarily on results from the 86 subjects who received the 3-step SUD dosing regimen in Arm A.

Of the 86 subjects treated in Arm A, a total of 49 (57.0%) were male. The median age was 63.5 years (range: 33, 90), with 39 (45.3%) subjects ≥65 years old and 10 (11.6%) subjects ≥75 years old. Sixty-four (74.4%) subjects were White, 2 (2.3%) subjects were Asian (both were Korean), 1 (1.2%) subject was Black or African American, and 19 (22.1%) subjects did not have race reported due to country-specific data protection laws.

Subjects in Arm A had highly refractory and high-risk FL disease. Advanced stage (Ann Arbor stage III and IV) disease was present in 79 (91.9%) subjects at trial entry and 44 (51.2%) subjects had a FLIPI score ≥3. Fifty-four (62.8%) subjects were double refractory to anti-CD20 and an alkylating agent, and 42 (48.8%) subjects experienced POD24. The median number of prior lines of systemic anti-lymphoma therapy was 2.0 (range: 2, 9), and 17 (19.8%) subjects received 4 or more prior lines of anti-lymphoma therapies.

Efficacy was not a primary endpoint for the Optimization Part of GCT3013-01; however, the ORR (CR+PR) and CR rates were assessed by the investigator using Lugano criteria as secondary endpoints (Error! Reference source not found.). After a median follow-up of 5.7 months (range: 0.4, 11.8), the ORR in subjects in Arm A was 86.0% (95% CI: 76.9, 92.6), and the CR rate was 64.0% (95% CI: 52.9, 74.0). In addition, 35 (44.9%) of the 78 subjects with tumor assessments at Week 6 had a CR; 26 (33.3%) had a PR. At Week 12, 39 (65.0%) of the 60 subjects with a tumor assessment had a CR and 10 (16.7%) had a PR. These data are consistent with efficacy results observed in subjects with FL in the pivotal cohort who received the epcoritamab 0.16/0.8/48 mg 2-step SUD dosing regimen.

In subjects in Arm A, the median TTR was 1.4 months (range: 1.2, 4.4) and the median TTCR was 1.5 months (range: 1.2, 4.7).

TABLE 11
Summary of Sequences

| SEQ | | |
|---|---|---|
| ID | Description | Sequence |
| 1 | huCD3 VH CDR1 | GFTFNTYA |
| 2 | huCD3 VH CDR2 | IRSKYNNYAT |
| 3 | huCD3 VH CDR3 | VRHGNFGNSYVSWFAY |
| 4 | huCD3 VL CDR1 | TGAVTTSNY |
| — | huCD3 VL CDR2 | GTN |
| 5 | huCD3 VL CDR3 | ALWYSNLWV |
| 6 | huCD3 VH1 | EVKLVESGGGLVQPGGSLRLSCAASGFTFNTYAMNWVRQAPGKGLE |
| | | WVARIRSKYNNYATYYADSVKDRFTISRDDSKSSLYLQMNNLKTEDTA |
| | | MYYCVRHGNFGNSYVSWFAYWGQGTLVTVSS |
| 7 | huCD3 VL1 | QAVVTQEPSFSVSPGGTVTLTCRSSTGAVTTSNYANWVQQTPGQAF |
| | | RGLIGGTNKRAPGVPARFSGSLIGDKAALTITGAQADDESIYFCALWYS |
| | | NLWVFGGGTKLTVL |
| 8 | VH CD20-7D8 CDR1 | GFTFHDYA |
| 9 | VH CD20-7D8 CDR2 | ISWNSGTI |
| 10 | VH CD20-7D8 CDR3 | AKDIQYGNYYYGMDV |
| 11 | VL CD20-7D8 CDR1 | QSVSSY |
| — | VL CD20-7D8 CDR2 | DAS |
| 12 | VL CD20-7D8 CDR3 | QQRSNWPIT |
| 13 | VH CD20-7D8 | EVQLVESGGGLVQPDRSLRLSCAASGFTFHDYAMHWVRQAPGKGLE |
| | | WVSTISWNSGTIGYADSVKGRFTISRDNAKNSLYLQMNSLRAEDTAL |
| | | YYCAKDIQYGNYYYGMDVWGQGTTVTVSS |
| 14 | VL CD20-7D8 | EIVLTQSPATLSLSPGERATLSCRASQSVSSYLAWYQQKPGQAPRLLIY |
| | | DASNRATGIPARFSGSGSGTDFTLTISSLEPEDFAVYYCQQRSNWPITF |
| | | GQGTRLEIK |
| 15 | IgG1 heavy chain | ASTKGPSVFPLAPSSKSTSGGTAALGCLVKDYFPEPVTVSWNSGALTS |
| | constant region-WT | GVHTFPAVLQSSGLYSLSSVVTVPSSSLGTQTYICNVNHKPSNTKVDK |
| | (amino acids positions | RVEPKSCDKTHTCPPCPAPELLGGPSVFLFPPKPKDTLMISRTPEVTCV |
| | 118-447 according to | VVDVSHEDPEVKFNWYVDGVEVHNAKTKPREEQYNSTYRVVSVLTV |
| | EU numbering). | LHQDWLNGKEYKCKVSNKALPAPIEKTISKAKGQPREPQVYTLPPSRE |
| | CH3 region italics | EMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 16 | IgG1-LFLEDA heavy | ASTKGPSVFPLAPSSKSTSGGTAALGCLVKDYFPEPVTVSWNSGALTS |
| | chain constant region | GVHTFPAVLQSSGLYSLSSVVTVPSSSLGTQTYICNVNHKPSNTKVDK |
| | (amino acids positions | RVEPKSCDKTHTCPPCPAPEFEGGPSVFLFPPKPKDTLMISRTPEVTCV |
| | 118-447 according to | VVAVSHEDPEVKFNWYVDGVEVHNAKTKPREEQYNSTYRVVSVLTV |
| | EU numbering). | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 17 | IgG1 F405L | ASTKGPSVFPLAPSSKSTSGGTAALGCLVKDYFPEPVTVSWNSGALTS |
| | (amino acids positions | GVHTFPAVLQSSGLYSLSSVVTVPSSSLGTQTYICNVNHKPSNTKVDK |
| | 118-447 according to | RVEPKSCDKTHTCPPCPAPELLGGPSVFLFPPKPKDTLMISRTPEVTCV |
| | EU numbering) | VVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FLLYSKLTVDKSRWQQGNVFSCSVMHEALHNHYTQKSLSLSPG |
| 18 | IgG1-K409R | ASTKGPSVFPLAPSSKSTSGGTAALGCLVKDYFPEPVTVSWNSGALTS |
| | (amino acids positions | GVHTFPAVLQSSGLYSLSSVVTVPSSSLGTQTYICNVNHKPSNTKVDK |
| | 118-447 according to | RVEPKSCDKTHTCPPCPAPELLGGPSVFLFPPKPKDTLMISRTPEVTCV |
| | EU numbering) | VVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSRLTVDKSRWQQGNVFSCSVMHEALHNHYTQKSLSLSPG |
| 19 | IgG1-LFLEDA-F405L | ASTKGPSVFPLAPSSKSTSGGTAALGCLVKDYFPEPVTVSWNSGALTS |
| | (FEAL) | GVHTFPAVLQSSGLYSLSSVVTVPSSSLGTQTYICNVNHKPSNTKVDK |
| | (amino acids positions | RVEPKSCDKTHTCPPCPAPEFEGGPSVFLFPPKPKDTLMISRTPEVTCV |
| | 118-447 according to | VVAVSHEDPEVKFNWYVDGVEVHNAKTKPREEQYNSTYRVVSVLTV |
| | EU numbering) | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FLLYSKLTVDKSRWQQGNVFSCSVMHEALHNHYTQKSLSLSPG |
| 20 | IgG1-LFLEDA-K409R | ASTKGPSVFPLAPSSKSTSGGTAALGCLVKDYFPEPVTVSWNSGALTS |
| | (FEAR) | GVHTFPAVLQSSGLYSLSSVVTVPSSSLGTQTYICNVNHKPSNTKVDK |
| | (amino acids positions | RVEPKSCDKTHTCPPCPAPEFEGGPSVFLFPPKPKDTLMISRTPEVTCV |
| | 118-447 according to | VVAVSHEDPEVKFNWYVDGVEVHNAKTKPREEQYNSTYRVVSVLTV |
| | EU numbering) | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSRLTVDKSRWQQGNVFSCSVMHEALHNHYTQKSLSLSPG |
| 21 | IgG1 CH3 region | GQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALH |
| | | NHYTQKSLSLSPG |
| 22 | Constant region | GQPKAAPSVTLFPPSSEELQANKATLVCLISDFYPGAVTVAWKADSSP |
| | human lambda LC | VKAGVETTTPSKQSNNKYAASSYLSLTPEQWKSHRSYSCQVTHEGST |
| | | VEKTVAPTECS |
| 23 | Constant region | RTVAAPSVFIFPPSDEQLKSGTASVVCLLNNFYPREAKVQWKVDNAL |
| | human kappa LC | QSGNSQESVTEQDSKDSTYSLSSTLTLSKADYEKHKVYACEVTHQGLS |
| | | SPVTKSFNRGEC |
| 24 | huCD3-LFLEDA-F405L | EVKLVESGGGLVQPGGSLRLSCAASGFTFNTYAMNWVRQAPGKGLE |
| | (FEAL) | WVARIRSKYNNYATYYADSVKDRFTISRDDSKSSLYLQMNNLKTEDTA |
| | heavy chain | MYYCVRHGNFGNSYVSWFAYWGQGTLVTVSSASTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTCPPC |
| | | PAPEFEGGPSVFLFPPKPKDTLMISRTPEVTCVVVAVSHEDPEVKFNW |
| | | YVDGVEVHNAKTKPREEQYNSTYRVVSVLTVLHQDWLNGKEYKCKV |
| | | SNKALPAPIEKTISKAKGQPREPQVYTLPPSREEMTKNQVSLTCLVKGF |
| | | YPSDIAVEWESNGQPENNYKTTPPVLDSDGSFLLYSKLTVDKSRWQQ |
| | | GNVFSCSVMHEALHNHYTQKSLSLSPG |
| 25 | huCD3 VL + CL light | QAVVTQEPSFSVSPGGTVTLTCRSSTGAVTTSNYANWVQQTPGQAF |
| | chain | RGLIGGTNKRAPGVPARFSGSLIGDKAALTITGAQADDESIYFCALWYS |
| | | NLWVFGGGTKLTVLGQPKAAPSVTLFPPSSEELQANKATLVCLISDFY |
| | | PGAVTVAWKADSSPVKAGVETTTPSKQSNNKYAASSYLSLTPEQWKS |
| | | HRSYSCQVTHEGSTVEKTVAPTECS |
| 26 | CD20-7D8-LFLEDA- | EVQLVESGGGLVQPDRSLRLSCAASGFTFHDYAMHWVRQAPGKGLE |
| | K409R (FEAR) | WVSTISWNSGTIGYADSVKGRFTISRDNAKNSLYLQMNSLRAEDTAL |
| | heavy chain | YYCAKDIQYGNYYYGMDVWGQGTTVTVSSASTKGPSVFPLAPSSKST |
| | | SGGTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPCPA |
| | | PEFEGGPSVFLFPPKPKDTLMISRTPEVTCVVVAVSHEDPEVKFNWYV |
| | | DGVEVHNAKTKPREEQYNSTYRVVSVLTVLHQDWLNGKEYKCKVSN |
| | | KALPAPIEKTISKAKGQPREPQVYTLPPSREEMTKNQVSLTCLVKGFYP |
| | | SDIAVEWESNGQPENNYKTTPPVLDSDGSFFLYSRLTVDKSRWQQG |
| | | NVFSCSVMHEALHNHYTQKSLSLSPG |
| 27 | CD20-7D8 VL + CL | EIVLTQSPATLSLSPGERATLSCRASQSVSSYLAWYQQKPGQAPRLLIY |
| | light chain | DASNRATGIPARFSGSGSGTDFTLTISSLEPEDFAVYYCQQRSNWPITF |
| | | GQGTRLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACE |
| | | VTHQGLSSPVTKSFNRGEC |
| 28 | Human CD3 (epsilon) | MQSGTHWRVLGLCLLSVGVWGQDGNEEMGGITQTPYKVSISGTTVI |
| | | LTCPQYPGSEILWQHNDKNIGGDEDDKNIGSDEDHLSLKEFSELEQSG |
| | | YYVCYPRGSKPEDANFYLYLRARVCENCMEMDVMSVATIVIVDICITG |
| | | GLLLLVYYWSKNRKAKAKPVTRGAGAGGRQRGQNKERPPPVPNPDY |
| | | EPIRKGQRDLYSGLNQRRI |
| 29 | Human CD20 | MTTPRNSVNGTFPAEPMKGPIAMQSGPKPLFRRMSSLVGPTQSFFM |
| | | RESKTLGAVQIMNGLFHIALGGLLMIPAGIYAPICVTVWYPLWGGIM |
| | | YIISGSLLAATEKNSRKCLVKGKMIMNSLSLFAAISGMILSIMDILNIKIS |
| | | HFLKMESLNFIRAHTPYINIYNCEPANPSEKNSPSTQYCYSIQSLFLGILS |
| | | VMLIFAFFQELVIAGIVENEWKRTCSRPKSNIVLLSAEEKKEQTIEIKEEV |
| | | VGLTETSSQPKNEEDIEIIPIQEEEEEETETNFPEPPQDQESSPIENDSSP |

Bold and underlined are FE; A; L and R, corresponding to positions 234 and 235; 265; 405 and 409, respectively, said positions being in accordance with EU-numbering. In variable regions, said CDR regions that were annotated in accordance with IMGT definitions are underlined.

1. A method of treating lymphoma in a human subject, the method comprising administering to the subject epcoritamab
wherein the epcoritamab is administered subcutaneously in a 28 day cycle comprising administering a priming dose on day 1 of about 0.05 mg to about 0.35 mg, a first intermediate dose on about day 8 of about 0.6 mg to 5 mg, a second intermediate dose on about day 15 of about 1 mg to about 10 mg, followed by at least one weekly dose of between about 20-100 mg; or
wherein the epcoritamab is administered subcutaneously in a 35 day cycle comprising administering a priming dose on day 1 of about 0.05 mg to about 0.35 mg, a first intermediate dose on about day 8 of about 0.6 mg to 5 mg, a second intermediate dose on about day 15 of about 1 mg to about 10 mg, followed by at least two weekly doses of between about 20-100 mg,
wherein after the 28 day or 35 day cycle, the epcoritamab is administered once a week, once every two weeks, once every four weeks or a combination thereof to the subject.

2. The method of claim 1, wherein the priming dose is about 0.16 mg.

3. The method of claim 1, wherein the first intermediate dose is about 0.6-1.2 mg.

4. The method of claim 3, wherein the first intermediate dose is about 0.8 mg.

5. The method of claim 1, wherein the second intermediate dose is about 3-6 mg.

6. The method of claim 5, wherein the second intermediate dose is 3 mg.

7. The method of claim 5, wherein the second intermediate dose is 6 mg.

8. The method of claim 1, wherein the weekly dose is between about 20 to 60 mg.

9. The method of claim 8, wherein the weekly dose is about 24 mg.

10. The method of claim 8, wherein the weekly dose is about 48 mg.

11. The method of claim 1, wherein the lymphoma is indolent lymphoma selected from follicular lymphoma (FL), cutaneous T-cell lymphoma (CTCL), marginal zone lymphoma (MZL), chronic lymphocytic leukemia (CLL) or small cell lymphocytic lymphoma (SLL), and/or wherein the aggressive lymphoma is large B-cell lymphoma (LBCL), such as Diffuse large B-cell lymphoma (DLBCL), high-grade B-cell lymphoma (HGBCL), primary mediastinal large B-cell lymphoma (PMBCL), follicular lymphoma (FL) grade 3B and mantle cell lymphoma (MCL).

12. The method of claim 11, wherein the indolent lymphoma is FL.

13. The method of claim 1, wherein the subject has received prior anti-neoplastic therapy.

14. The method of claim 13, wherein the subject has received prior treatment with a CD20 monospecific antibody.

15. The method of claim 11, wherein the indolent lymphoma is relapsed or refractory.

16. The method of claim 1, wherein the weekly administration is performed at least 4 times.

17. The method of claim 1, wherein after the weekly administration, the coritamab is administered once every two weeks.

18. The method of claim 17, wherein administration once every two weeks of the epcoritamab is performed at least six (6) times.

19. The method of claim 17, wherein after the biweekly administration, the epcoritamab is administered once every four weeks.

20. (canceled)

21. The method of claim 1, comprising:
(a) a first cycle of 28-days wherein
(i) the priming dose of the epcoritamab is administered on Day 1;
(ii) the first intermediate dose of the epcoritamab is administered on Day 8;
(iii) the second intermediate dose of the epcoritamab is administered on Day 15;
(iv) a dose of 24 mg of the epcoritamab is administered on Day 22; and
(b) cycles 2-3 each comprising 28 days wherein a dose of 24 mg of the epcoritamab is administered on Days 1, 8, 15 and 22;
(c) cycles 4-9 each comprising 28 days wherein a dose of 24 mg of the bispecific antibody epcoritamab is administered on Days 1 and 15; and
(d) further subsequent 28 day cycles, wherein a dose of 24 mg of the epcoritamab is administered on Day 1.

22. The method of claim 1, comprising:
(a) a first cycle of 28-days wherein
(i) the priming dose of the epcoritamab is administered on Day 1;
(ii) the first intermediate dose of the epcoritamab is administered on Day 8;
(iii) the second intermediate dose of the epcoritamab is administered on Day 15;
(iv) a dose of 48 mg of the epcoritamab is administered on Day 22; and
(b) cycles 2-3 each comprising 28 days wherein a dose of 48 mg of the epcoritamab is administered on Days 1, 8, 15 and 22;
(c) cycles 4-9 each comprising 28 days wherein a dose of 48 mg of the epcoritamab is administered on Days 1 and 15; and
(d) further subsequent 28 day cycles, wherein a dose of 48 mg of the epcoritamab is administered on Day 1.

23. The method of claim 1, comprising:
(a) a first cycle of 28-days wherein
(i) a priming dose of 0.16 mg of the epcoritamab is administered on Day 1;
(ii) a first intermediate dose 0.8 mg of the epcoritamab is administered on Day 8;
(iii) a second intermediate dose of 3 mg of the epcoritamab is administered on Day 15;
(iv) a dose of 48 mg of the epcoritamab is administered on Day 22; and
(b) cycles 2-3 each comprising 28 days wherein a dose of 48 mg of the epcoritamab is administered on Days 1, 8, 15 and 22;
(c) cycles 4-9 each comprising 28 days wherein a dose of 48 mg of the epcoritamab is administered on Days 1 and 15; and
(d) further subsequent 28 day cycles, wherein a dose of 48 mg of the epcoritamab is administered on Day 1.

24. The method of claim 1, comprising:
(a) a first cycle of 28-days wherein
(i) a priming dose of 0.16 mg of the epcoritamab is administered on Day 1;
(ii) a first intermediate dose 0.8 mg of the epcoritamab is administered on Day 8;
(iii) a second intermediate dose of 6 mg of the epcoritamab is administered on Day 15;
(iv) a dose of 48 mg of the epcoritamab is administered on Day 22; and
(b) cycles 2-3 each comprising 28 days wherein a dose of 48 mg of the epcoritamab is administered on Days 1, 8, 15 and 22;
(c) cycles 4-9 each comprising 28 days wherein a dose of 48 mg of the epcoritamab is administered on Days 1 and 15; and
(d) further subsequent 28 day cycles, wherein a dose of 48 mg of the epcoritamab is administered on Day 1.

25. The method of claim 21, wherein the lymphoma is FL.

26. The method of claim 1, wherein the subject has manageable cytokine release syndrome (CRS) of grade 1 or grade 2 during administration of the epcoritamab.

27. The method of claim 1, wherein the subject does not experience tumor lysis syndrome.

28. The method of claim 1, wherein the subject is treated with prophylaxis for CRS.

29. The method of claim 28, wherein the prophylaxis includes the administration of a corticosteroid.

30. The method of claim 29, wherein the corticosteroid is dexamethasone.

31. The method of claim 30, wherein the dexamethasone is administered at a dose of about 2-20 mg.

32. The method of claim 31, wherein the dexamethasone is administered at a dose of about 15 mg.

33. The method of claim 28, wherein the prophylaxis is administered at the same day as the epcoritamab.

34. The method claim 33, wherein the prophylaxis is administered at subsequent days 2-3, and optionally day 4, or at subsequent days 2-4.

35. The method of claim 28, wherein when the prophylaxis is administered at the same day as the epcoritamab, the prophylaxis is administered 30-120 minutes prior to the administration of the epcoritamab.

36. The method of claim 1, wherein the human subject is treated with premedication to reduce reactions to injections.

37. The method of claim 36, wherein the premedication includes the administration of antihistamines.

38. The method of claim 36, wherein the premedication includes the administration of antipyretics.

39. The method of claim 37, wherein the antihistamine is diphenhydramine.

40. The method of claim 38, wherein the antipyretic is acetaminophen.

41. The method of claim 36, wherein the premedication is administered at the same day as the epcoritamab.

42. The method of claim 41, wherein the premedication administered 30-120 minutes prior to the administration of the epcoritamab.

43. The method of claim 28, wherein the prophylaxis is administered during the first cycle.

44. The method of claim 36, wherein the premedication is administered during the first cycle.

45. The method of claim 43, wherein the prophylaxis is administered during the second cycle when the human subject experiences CRS greater than grade 1 after the fourth administration of the epcoritamab in cycle 1.

46. The method of claim 28, wherein the prophylaxis is continued during a subsequent cycle, when in the last administration of the epcoritamab of the previous cycle, the human subject experiences CRS greater than grade 1.

47. The method of claim 38, wherein the premedication is optionally administered during the second cycle.

48. The method of claim 47, wherein the premedication is optionally administered during subsequent cycles.

49-63. (canceled)

64. The method of claim 1, wherein the lymphoma is aggressive.

65. The method of claim 64, wherein the aggressive lymphoma is large B-cell lymphoma (LBCL), Diffuse large B-cell lymphoma (DLBCL), high-grade B-cell lymphoma (HGBCL), primary mediastinal large B-cell lymphoma (PMBCL), follicular lymphoma (FL) grade 3B, mantle cell lymphoma (MCL).

66. The method of claim 39, wherein the diphenhydramine is administered at an intravenous or oral dose 50 mg, or equivalent thereof.

67. The method of claim 40, wherein the acetaminophen is administered at an oral dose of 650-1000 mg, or equivalent thereof
